(19) United States
(12) Patent Application Publication
(10) Pub. No.: US 2024/0293421 A1
(43) Pub. Date: Sep. 5, 2024

(54) CRYSTALLINE COMPOSITION OF TILDACERFONT AND METHODS OF USE AND PREPARATION THEREOF

(71) Applicant: Spruce Biosciences, Inc., South San Francisco, CA (US)

(72) Inventors: Dasharatha REDDY, San Ramon, CA (US); Ashokraj RAJAGOPAL, Alameda, CA (US); Lu WANG, Berkeley, CA (US); Christopher BARNES, San Francisco, CA (US)

(21) Appl. No.: 18/598,559

(22) Filed: Mar. 7, 2024

Related U.S. Application Data
(63) Continuation of application No. PCT/US2022/050436, filed on Nov. 18, 2022.
(60) Provisional application No. 63/340,874, filed on May 11, 2022.
(60) Provisional application No. 63/281,462, filed on Nov. 19, 2021.

(51) Int. Cl. A61K 31/5377; A61K 47/12

(52) U.S. Cl. CPC A61K 31/5377; A61K 47/12

(57) ABSTRACT

Described herein are crystalline composition, methods of making such crystalline composition, pharmaceutical compositions and medicaments comprising such crystalline composition, and methods of using such crystalline composition in the treatment of conditions, diseases, or disorders that would benefit from modulation of Corticotropin-releasing hormone receptor 1 (CRF1).

### CROSS-REFERENCE

This application is a continuation of International Application No. PCT/US2022/050436 filed on Nov. 18, 2022, which claims the benefit of U.S. Provisional Application No. 63/281,462 filed Nov. 19, 2021, and U.S. Provisional Application No. 63/340,874 filed May 11, 2022, each of which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Corticotropin releasing factor (CRF) is a 41 amino acid peptide that is the primary physiological regulator of proopiomelanocortin (POMC) derived peptide secretion from the anterior pituitary gland. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in the brain. There is also evidence that CRF plays a significant role in integrating the response in the immune system to physiological, psychological, and immunological stressors.

### SUMMARY

In a first aspect, the present disclosure provides a crystalline composition comprising Compound I.

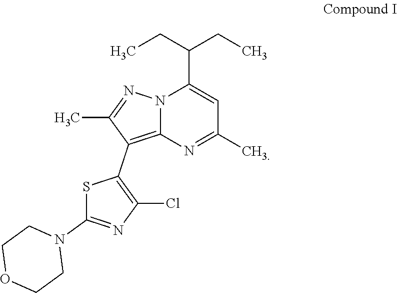 [SMILES: CCC(CC)c1cc(C)nc2c(-c3sc(N4CCOCC4)nc3Cl)c(C)nn12]

In some embodiments, the crystalline composition comprising Compound 1 comprises a crystalline composition comprising Formula I:

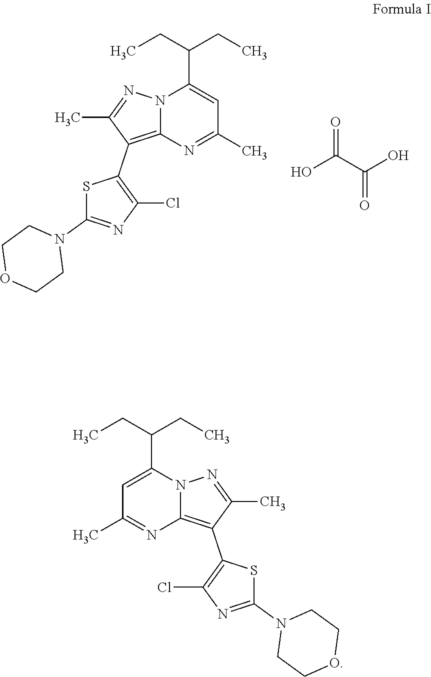

In some embodiments, the crystals of the crystalline composition comprising Compound I (e.g., comprising Formula I) have unit cell parameters at T=150° K of: a=34.003(4) Å, b=6.5843(13) Å, c=21.062(5) Å; β=108.703(12)°, V=4466.5(15) Å³ and a monoclinic C2/c space group.

In some embodiments, the crystals of the crystalline composition comprising Compound I (e.g., comprising Formula I) is characterized by:
(a) an X-ray powder diffraction pattern comprising peaks at 10.84±0.2° 2-θ, 15.96±0.2° 2-θ, 23.44±0.2° 2-θ, and 24.80±0.2° 2-θ, and as measured by X-ray powder diffraction using an X-ray wavelength of 0.71073 Å;
(b) an X-ray powder diffraction pattern substantially the same as shown in FIG. 1;
(c) a differential scanning calorimetry (DSC) thermogram comprising an endotherm in the range of about 160° C. to 170° C.;
(d) a differential scanning calorimetry (DSC) thermogram comprising an endotherm with an onset of about 164° C. and a peak of about 166° C.;
(e) a differential scanning calorimetry (DSC) thermogram substantially the same as shown in FIG. 2;
(f) a thermogravimetric analysis (TGA) thermogram comprising a loss in mass of about 11% over a temperature range of about 25° C. to about 200° C.;
(g) a thermogravimetric analysis (TGA) thermogram substantially the same as shown in FIG. 3; or
(h) combinations thereof.

In some embodiments, the crystals of the crystalline composition comprising Compound I (e.g., comprising Formula I) is characterized by (a), (c), (d), and (f).

In some embodiments, the composition comprising a CRF1 antagonist (e.g., a crystalline composition comprising Compound I (e.g., comprising Formula I)) is characterized by an X-ray powder diffraction pattern comprising one or more peaks at 10.84±0.2° 2-θ, 15.96±0.2° 2-θ, 23.44±0.2° 2-θ, and 24.80±0.2° 2-θ, and as measured by X-ray powder diffraction using an X-ray wavelength of 0.71073 Å. In some embodiments, the CRF1 antagonist (e.g., crystalline composition comprising Compound I (e.g., comprising Formula I)) is characterized by an X-ray powder diffraction pattern comprising peaks at 10.84±0.2° 2-θ, 15.96±0.2° 2-θ, 23.44±0.2° 2-θ, and 24.80±0.2° 2-θ, and as measured by X-ray powder diffraction using an X-ray wavelength of 0.71073 Å. In some embodiments, the X-ray powder diffraction pattern further comprising at least one peak selected from 5.44±0.2° 2-θ, 20.78±0.2° 2-θ, 22.74±0.2° 2-θ, 23.04±0.2° 2-θ, 26.80±0.2° 2-θ, and 28.86±0.2° 2-θ, and as measured by X-ray powder diffraction using an X-ray wavelength of 0.71073 Å. In some embodiments, the X-ray powder diffraction pattern further comprising at least five peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the crystalline composition is characterized by an X-ray powder diffraction pattern substantially the same as shown in FIG. 1.

In some embodiments, the crystalline composition comprising a CRF1 antagonist (e.g., crystalline composition comprising Compound I (e.g., comprising Formula I)) is characterized by a differential scanning calorimetry (DSC) thermogram comprising an endotherm in the range of about 160° C. to 170° C. In some embodiments, the crystalline composition is characterized by a differential scanning calorimetry (DSC) thermogram comprising an endotherm with an onset of about 164° C. and a peak of about 166° C. In some embodiments, the crystalline composition is characterized by a DSC thermogram comprising an endotherm with an onset from about 160° C. to about 170° C. In some embodiments, the crystalline composition is characterized by a DSC thermogram comprising an endotherm with an onset from about 150° C. to about 170° C. In some embodiments, the crystalline composition is characterized by a DSC thermogram comprising an endotherm with an onset from about 160° C. to about 180° C. In some embodiments, the crystalline composition is characterized by a DSC thermogram comprising an endotherm with an onset at about 160° C. In some embodiments, the crystalline composition is characterized by a DSC thermogram comprising a peak of about 166° C. In some embodiments, the crystalline composition is characterized by a DSC thermogram comprising a peak from about 166° C. In some embodiments, the crystalline composition is characterized by a melting point of about 166° C. In some embodiments, the crystalline composition is characterized by a melting point of about 160° C. to about 170° C. In some embodiments, the crystalline composition is characterized by a melting point of about 150° C. to about 170° C. In some embodiments, the crystalline composition is characterized by a melting point of about 160° C. to about 180° C. In some embodiments, the crystalline composition is characterized by a differential scanning calorimetry (DSC) thermogram substantially the same as shown in FIG. 2.

In some embodiments, the crystalline composition comprising a CRF1 antagonist (e.g., crystalline composition comprising Compound I (e.g., comprising Formula I)) is characterized by a thermogravimetric analysis (TGA) thermogram comprising a loss in mass of about 11% over a temperature range of about 25° C. to about 200° C. In some embodiments, the crystalline composition is characterized by a thermogravimetric analysis (TGA) thermogram substantially the same as shown in FIG. 3. In some embodiments, the crystalline composition is characterized by a TGA thermogram comprising a loss in mass of more than 10% over a temperature range of about 25° C. to about 200° C. In some embodiments, the crystalline composition is characterized by a TGA thermogram comprising a loss in mass of less than about 15% over a temperature range of about 25° C. to about 200° C. In some embodiments, the crystalline composition is characterized by a TGA thermogram comprising a loss in mass of less than about 20% over a temperature range of about 25° C. to about 200° C. In some embodiments, the crystalline composition is characterized by a dynamic vapor sorption (DVS) trace substantially the same as shown in FIG. 4.

In some aspects, the present disclosure provides a pharmaceutical composition comprising crystalline composition comprising Formula I:

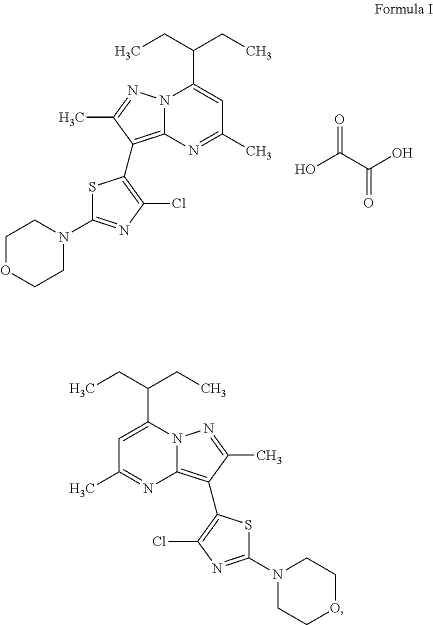

and at least one pharmaceutically acceptable carrier or excipient.

In some aspects, the present disclosure provides a pharmaceutical composition comprising Compound 1:

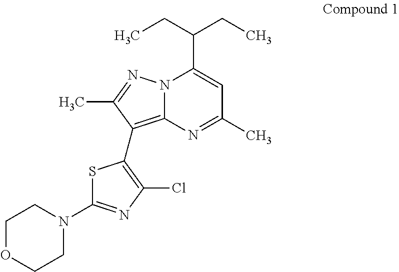 [SMILES: CCC(CC)c1cc(C)nc2c(-c3sc(N4CCOCC4)nc3Cl)c(C)nn12]

or a pharmaceutically acceptable salt thereof, and at least one pharmaceutically acceptable carrier or excipient. In some embodiments, the pharmaceutical composition comprising Compound 1 is a crystalline pharmaceutical composition. In some embodiments, the pharmaceutical composition comprises crystalline Compound 1.

In another aspect, the present disclosure provides a method of preparing a crystalline composition comprising Compound 1 (e.g., comprising Formula I:

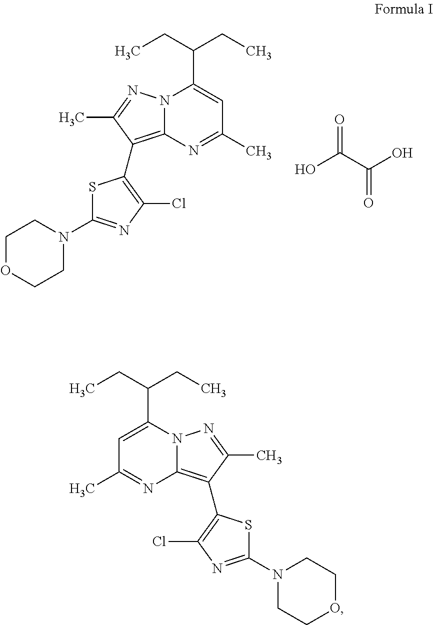

wherein the method comprises:
a) dissolving 4-(4-chloro-5-(2,5-dimethyl-7-(pentan-3-yl)pyrazolo[1,5-a]pyrimidin-3-yl)thiazol-2-yl)morpholine (Compound 1) and oxalic acid in a solvent;
(b) heating the solution or slurry from step (a); and
(c) crystallizing the solution or slurry obtained in step (b) to obtain the crystalline composition comprising Compound 1 (e.g., comprising Formula I).

In some embodiments of the method of preparing the crystalline composition comprising Compound I (e.g., comprising Formula I), the solvent in step (a) comprises acetone, heptane, water, 2-butanol, ethyl acetate, 2-propanol, methyl tert-butyl ether, ethanol, methyl ethyl ketone, 1-pentanol, or a combination thereof. In some embodiments, the solvent in step (a) is heptane. In some embodiments, the solvent in step (a) is methyl tert-butyl ether or methyl ethyl ketone. In some embodiments, the solvent in step (a) is a mixture of heptane and methyl ethyl ketone. In some embodiments, step (b) is heated at about 50° C.

In another aspect, the present disclosure provides a method of treating or preventing testicular adrenal rest tumors (TART) or ovarian adrenal rest tumors (OART) in a subject in need thereof, comprising administering to the subject a crystalline composition comprising Formula I as described herein, the pharmaceutical composition comprising the crystalline composition comprising Formula I as described herein, or the pharmaceutical composition comprising Compound 1 as described herein. In some embodiments, the subject has congenital adrenal hyperplasia (CAH).

In another aspect, the present disclosure provides a method of treating congenital adrenal hyperplasia (CAH) in a subject in need thereof, comprising administering to the subject a crystalline composition comprising Formula I as described herein, a pharmaceutical composition comprising the crystalline composition comprising Formula I as described herein, or the pharmaceutical composition comprising Compound 1 as described herein. In some embodiments, the CAH is a classic CAH. In some embodiments, the CAH is a non-classical CAH.

In another aspect, the present disclosure provides a method of improving (e.g., lessening the severity and/or frequency) hyperandrogenic symptoms in a subject in need thereof, comprising administering to the subject a crystalline composition comprising Formula I as described herein, the pharmaceutical composition comprising the crystalline composition comprising Formula I as described herein, or the pharmaceutical composition comprising Compound 1 as described herein. In some embodiments, the hyperandrogenic symptoms are selected from the group consisting of acne, hirsutism, amenorrhea, and alopecia.

In another aspect, the present disclosure provides a method of treating menstrual irregularity, ovulatory dysfunction or infertility, in a subject in need thereof, comprising administering to the subject a crystalline composition comprising Formula I as described herein, the pharmaceutical composition comprising the crystalline composition comprising Formula I as described herein, or the pharmaceutical composition comprising Compound 1 as described herein.

In another aspect, the present disclosure provides a method of improving (e.g., lessening the severity and/or frequency) metabolic symptoms in a subject in need thereof, comprising administering to the subject a crystalline composition comprising Formula I as described herein, the pharmaceutical composition comprising the crystalline composition comprising Formula I as described herein, or the pharmaceutical composition comprising Compound 1 as described herein. In some embodiments, the metabolic symptoms are selected from the group consisting of body weight, BMI, fat mass, waist circumference, blood pressure and glycemic control.

Metabolic symptoms may be evaluated using an assessment, such as, for example, fasting lipid panel, homeostatic model assessment of insulin resistance [HOMA-IR] based on fasting glucose and insulin levels, glycated hemoglobin [HbAlc], glucose tolerance test.

In another aspect, the present disclosure provides a method of treating polycystic ovary syndrome with functional ovarian hyperandrogenism and functional adrenal hyperandrogenism (PCOS-FOH+FAH) in a subject in need thereof, comprising administering to the subject a crystalline composition comprising Formula I as described herein, the pharmaceutical composition comprising the crystalline composition comprising Formula I as described herein, or the pharmaceutical composition comprising Compound 1 as described herein.

In another aspect, the present disclosure provides a method of treating polycystic ovary syndrome with functional adrenal hyperandrogenism (PCOS-FAH) in a subject in need thereof, comprising administering to the subject a crystalline composition comprising Formula I as described herein, the pharmaceutical composition comprising the crystalline composition comprising Formula I as described herein, or the pharmaceutical composition comprising Compound 1 as described herein.

In another aspect, the present disclosure provides a method of treating endometriosis or improving the symptoms of endometriosis in a subject in need thereof, comprising administering to the subject a crystalline composition comprising Formula I as described herein, the pharmaceutical composition comprising the crystalline composition comprising Formula I as described herein, or the pharmaceutical composition comprising Compound 1 as described herein. In some embodiments, the administration of the crystalline composition or the pharmaceutical composition improves the symptom of pain experienced by the subject. In some embodiments, the administration of the crystalline composition or the pharmaceutical composition reduces the symptoms of pain experienced by the subject. In some embodiments, the administration of the crystalline composition or the pharmaceutical composition prevents or lowers the perception of pain experienced by the subject (e.g., as measured on a pain scale). In some embodiments, the administration of the crystalline composition or the pharmaceutical composition eliminates the perception of pain experienced by the subject.

In various embodiments of the methods as described herein, the crystalline composition or the pharmaceutical composition is administered at a dose from about 5 mg/day to about 1000 mg/day. In some embodiments, the crystalline composition or the pharmaceutical composition is administered in a fed state. A fed state may mean that the subject has eaten within a 30-minute period before of receiving the crystalline composition or the pharmaceutical compositions. In some embodiments, the crystalline composition or the pharmaceutical composition is administered in a fasted state. A fasted state may mean that the subject has not eaten within a 30-minute period before of receiving the crystalline composition or the pharmaceutical compositions. In some embodiments, the crystalline composition or the pharmaceutical composition is administered at least once per day. In some embodiments, the crystalline composition or the pharmaceutical composition is administered once a day. In some embodiments, the crystalline composition or the pharmaceutical composition is administered twice a day.

In various embodiments of the methods as described herein, the method further comprises administering to the subject an additional therapeutic agent. In some embodiments, the additional therapeutic agent is glucocorticoid, a mineralocorticoid, an ACAT1 inhibitor, or an anti-androgen agent. In some embodiments, the glucocorticoid is beclomethasone, betamethasone, budesonide, cortisone, dexamethasone, hydrocortisone, methylprednisolone, prednisolone, prednisone, or triamcinolone. In some embodiments, the mineralocorticoid is fludrocortisone. In some embodiments, the additional therapeutic agent is a different CRF1 antagonist.

In various embodiments of the methods as described herein, the method further comprises an additional treatment selected from surgical resection of the tumors and radiation therapy, or a combination thereof. In some embodiments, the additional therapy is surgical resection and the surgical resection is prior to, after, and/or concurrent with administration of the crystalline composition as described herein, or the pharmaceutical composition comprising the crystalline composition as described herein. In some embodiments, the additional therapy is radiation therapy and the radiation therapy is prior to, after, and/or concurrent with administration of the crystalline composition as described herein, the pharmaceutical composition comprising the crystalline composition as described herein, or the pharmaceutical composition comprising Compound 1 as described herein.

In various embodiments of the methods, the subject is from about 9 years of age to about 18 years of age. In some embodiments, the subject is from about 8 years of age to about 55 years of age.

In additional aspects, provided herein is a pharmaceutical composition comprising a CRF1 antagonist, wherein the composition is characterized by an X-ray powder diffraction pattern comprising a peak selected from any one or more of 10.84±0.2° 2-θ, 15.96±0.2° 2-θ, 23.44±0.2° 2-θ, and 24.80±0.2° 2-θ, and as measured by X-ray powder diffraction using an X-ray wavelength of 0.71073 Å. In some embodiments, the composition is characterized by X-ray powder diffraction pattern comprising peaks at 10.84±0.2° 2-θ, 15.96±0.2° 2-θ, 23.44±0.2° 2-θ, and 24.80±0.2° 2-0. In some embodiments, the X-ray powder diffraction pattern further comprises at least one peak selected from 5.44±0.2° 2-θ, 20.78±0.2° 2-θ, 22.74±0.2° 2-θ, 23.04±0.2° 2-θ, 26.80±0.2° 2-θ, and 28.86±0.2° 2-θ, and as measured by X-ray powder diffraction using an X-ray wavelength of 0.71073 Å. In some embodiments, the X-ray powder diffraction pattern further comprises at least five peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the pharmaceutical composition is further characterized by a thermogravimetric analysis (TGA) thermogram comprising a loss in mass of about 11% over a temperature range of about 25° C. to about 200° C. In some embodiments, the CRF1 antagonist comprises Compound 1:

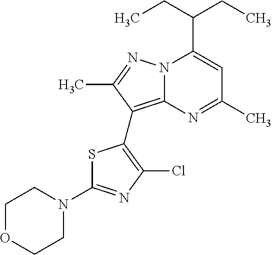 [SMILES: CCC(CC)c1cc(C)nc2c(-c3sc(N4CCOCC4)nc3Cl)c(C)nn12]

Compound 1, or a pharmaceutically acceptable salt thereof, and at least one pharmaceutically acceptable excipient. In some embodiments, the pharmaceutical composition is a crystalline pharmaceutical composition. In some embodiments, the crystalline pharmaceutical composition further comprises oxalic acid. In some embodiments, the crystalline pharmaceutical composition comprises Compound 1 and oxalic acid in a molar ratio of about 2:1.

In additional aspects, provided herein is a pharmaceutical composition comprising a CRF1 antagonist, wherein the pharmaceutical com-position is characterized by a differential scanning calorimetry (DSC) thermogram comprising an endotherm in the range of about 160° C. to 170° C. In some embodiments, the pharmaceutical composition is characterized by a differential scanning calorimetry (DSC) thermogram comprising an endotherm with an onset of about 164° C. and a peak of about 166° C. In some embodiments, the pharmaceutical composition is characterized by a melting point of about 166° C. In some embodiments, the CRF1 antagonist comprises Compound 1:

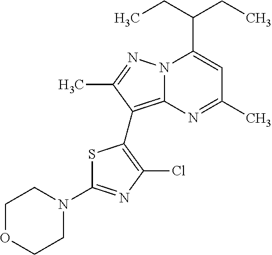 [SMILES: CCC(CC)c1cc(C)nc2c(-c3sc(N4CCOCC4)nc3Cl)c(C)nn12]

Compound 1, or a pharmaceutically acceptable salt thereof, and at least one pharmaceutically acceptable excipient. In some embodiments, the pharmaceutical composition is a crystalline pharmaceutical composition. In some embodiments, the crystalline pharmaceutical composition further comprises oxalic acid. In some embodiments, the crystalline pharmaceutical composition comprises Compound 1 and oxalic acid in a molar ratio of about 2:1. In some embodiments, the pharmaceutical composition is further characterized by a thermogravimetric analysis (TGA) thermogram comprising a loss in mass of about 11% over a temperature range of about 25° C. to about 200° C.

In additional aspects, provided herein is method of preparing a crystalline composition comprising Compound 1:

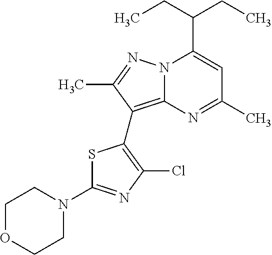 [SMILES: CCC(CC)c1cc(C)nc2c(-c3sc(N4CCOCC4)nc3Cl)c(C)nn12]

Compound 1, wherein the method comprises: (a) dissolving Compound 1 and an acid in a solvent; (b) heating the solution or slurry from step (a); and (c) crystallizing the solution or slurry obtained in step (b) to obtain the crystalline composition comprising Compound 1. In some embodiments, the acid comprises oxalic acid. In some embodiments, the solvent in step (a) is heptane. In some embodiments, the solvent in step (a) is methyl tert-butyl ether or methyl ethyl ketone. In some embodiments, the solvent in step (a) is a mixture of heptane and methyl ethyl ketone. In some embodiments, step (b) is heated at about 50° C.

In additional aspects, further provided herein is a pharmaceutical composition comprising Compound 1:

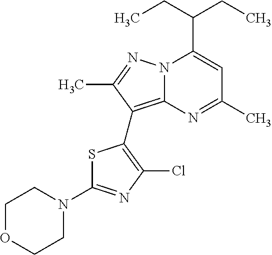 [SMILES: CCC(CC)c1cc(C)nc2c(-c3sc(N4CCOCC4)nc3Cl)c(C)nn12]

Compound 1, or a pharmaceutically acceptable salt or solvate thereof, wherein Compound 1 is present in an amount of 25% w/w; and a diluent in about 5% to about 70% In some embodiments, Compound 1 is in an amount of about 50 mg to about 200 mg. In some embodiments, Compound 1 is in an amount of about 50 mg. In some embodiments, wherein the diluent is selected from the group consisting of calcium carbonate, microcrystalline cellulose, calcium phosphate, starch, pregelatinized starch, sodium carbonate, mannitol, and lactose monohydrate, and a combination thereof. In some embodiments, the diluent is lactose monohydrate. In some embodiments, the lactose monohydrate is about 20% to about 45% by weight of the pharmaceutical composition. In some embodiments, the lactose monohydrate is about 20% to about 40.25% by weight of the pharmaceutical composition. In some embodiments, the lactose monohydrate is in an amount that is about 16.1, 40.25, 80.5, or 322 mg. In some embodiments, the diluent is microcrystalline cellulose. In some embodiments, the pharmaceutical composition further comprises a second diluent, wherein the second diluent comprises microcrystalline cellulose. In some embodiments, the microcrystalline cellulose is about 25% by weight of the pharmaceutical composition. In some embodiments, the microcrystalline cellulose is in an amount that is about 50 mg. In some embodiments, the pharmaceutical composition further comprises a binder, wherein the binder is selected from the group consisting of corn starch and potato starch, gelatin, sucrose hydroxypropyl cellulose (HPC), polyvinylpyrrolidone (PVP), and hydroxypropyl methyl cellulose (HPMC), and a combination thereof. In some embodiments, the binder comprises HPC. In some embodiments, the HPC is about 3% by weight of the solid dosage form. In some embodiments, the pharmaceutical composition further comprises a surfactant, wherein the surfactant is selected from the group consisting of sodium lauryl sulfate, tetradecyl trimethyl ammonium bromide with dodecyl, hexadecyl compound, benzalkonium chloride, cetylpyridinium chloride, alkylsulphate, alkylethoxylate sulphate, soap, carxylate ion, sulfate ion, sulfonate ion, polyoxyethylene derivative, polyoxypropylene derivative, polyol derivative, polyol ester, polyoxyethylene ester, poloxamers, glocol, glycerol ester, sorbitan derivative, and polyethylene glycol, and a combination thereof. In some embodiments, the surfactant comprises sodium lauryl sulfate. In some embodiments, the sodium lauryl sulfate is about 0.75% by weight of the solid dosage form. In some embodiments, the sodium lauryl sulfate is in an amount that is about 1.5 mg. In some embodiments, the pharmaceutical composition further comprises a disintegrant, wherein the disintegrant is selected from the group consisting of starch, alginic acid, ac-di-sol, croscarmellose sodium, sodium starch glycolate, and crospovidone, and a combination thereof. In some embodiments, the disintegrant comprises croscarmellose sodium.

In some embodiments, the pharmaceutical composition further comprises a glidant, wherein the glidant comprises colloidal silicon dioxide. In some embodiments, the colloidal silicon dioxide is about 1% by weight of the solid dosage form. In some embodiments, the colloidal silicon dioxide is in an amount that is about 2 mg. In some embodiments, the pharmaceutical composition further comprises a lubricant, wherein the lubricant is selected from the group consisting of talc, stearic acid, magnesium stearate, sodium stearyl fumarate, and a combination thereof. In some embodiments, the pharmaceutical formulation is in a solid dosage form. In some embodiments, the solid dosage form is a tablet. In some embodiments, the solid dosage form is a granules form. In some embodiments, said granules form is packaged in a capsule. In some embodiments, said granules form is packaged in a sachet.

In additional aspects, provided herein is a pharmaceutical formulation, comprising: (a) about 10 mg to about 300 mg of 3-(4-Chloro-2-(morpholin-4-yl)thiazol-5-yl)-7-(1-ethylpropyl)-2,5-dimethylpyrazolo(1,5-α) pyrimidine (Compound 1), or a pharmaceutically acceptable salt or solvate thereof; (b) a first diluent in about 40% to about 45% by weight, wherein the first diluent comprises lactose monohydrate; (c) a second diluent in about 25% by weight, wherein the second diluent comprises microcrystalline cellulose; (d) about 1% to about 6% by weight of hydroxypropyl cellulose; (e) about 0.5% to about 5% by weight of sodium lauryl sulfate; and (f) about 4% by weight of croscarmellose sodium.

### DESCRIPTION OF THE DRAWINGS

The novel features of the present disclosure are set forth with particularity in the appended claims. An understanding of the features and advantages of the present disclosure may be obtained by reference to the following detailed description that sets forth illustrative embodiments, in which the principles of the present disclosure are utilized, and the accompanying drawings of which:

FIG. 1 shows the X-ray powder diffraction (XRPD) pattern for the crystalline composition comprising Compound 1 (e.g., comprising Formula I).

FIG. 2 shows the differential scanning calorimetry (DSC) thermogram for the crystalline composition comprising Compound 1 (e.g., comprising Formula I).

FIG. 3 shows the thermogravimetric analysis (TGA) thermogram for the crystalline composition comprising Compound 1 (e.g., comprising Formula I).

FIG. 4 shows the dynamic vapor sorption (DVS) trace for the crystalline composition comprising Compound 1 (e.g., comprising Formula I).

FIG. 5 shows the infrared (IR) spectrum for the crystalline composition comprising Compound 1 (e.g., comprising Formula I).

FIG. 6 shows the proton nuclear magnetic resonance (¹H NMR) spectrum for the crystalline composition comprising Compound 1 (e.g., comprising Formula I).

FIG. 7 shows the Raman spectrum for the crystalline composition comprising Compound 1 (e.g., comprising Formula I).

### DETAILED DESCRIPTION

4-(4-chloro-5-(2,5-dimethyl-7-(pentan-3-yl)pyrazolo[1,5-a]pyrimidin-3-yl)thiazol-2-yl)morpholine (Compound 1) is a potent antagonist of Corticotropin-releasing hormone receptor 1 (CRF1). CRF1 antagonists may be useful in the treatment of various diseases, conditions, and disorders for which abnormal CRF1 activity plays a role.

Crystalline forms of a small molecule drug candidate, such as a CRF1 antagonist, may have different physical properties, including melting point, apparent solubility, dissolution rate, optical and mechanical properties, vapor pressure, and density. These properties may have an effect on the ability to process or manufacture a drug substance and the drug product. Moreover, differences in these properties can and often lead to different pharmacokinetics profiles for different polymorphic forms of a drug. Polymorphism may affect the quality, safety, and/or efficacy of a drug product, such as a CRF1 antagonist. Thus, there still remains a need for crystalline compositions of CRF1 antagonist. The present disclosure addresses this need and provides related advantages as well.

### Compound 1

As described herein, Compound 1 refers to 4-(4-chloro-5-(2,5-dimethyl-7-(pentan-3-yl)pyrazolo[1,5-a]pyrimidin-3-yl)thiazol-2-yl)morpholine, which has the chemical structure as shown below:

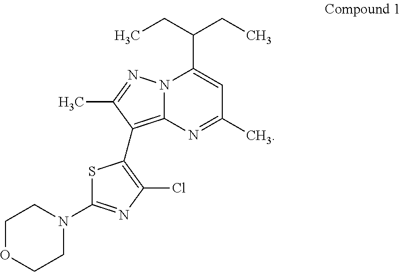 [SMILES: CCC(CC)c1cc(C)nc2c(-c3sc(N4CCOCC4)nc3Cl)c(C)nn12]

Compound 1 is a CRF1 modulator. CRF1 modulator, such as a CRF1 antagonist or inhibitor, are useful in the treatment of various conditions and disorders that are mediated by CRF1 activities.

In some embodiments, the present disclosure provides a crystalline composition comprising Compound 1. In some embodiments, the crystalline composition comprises Compound 1 and a weak acid (e.g., oxalic acid) (e.g., in a molar ratio of 2:1). In some embodiments, the crystalline composition comprises Compound 1 and an acid in a molar ratio of 2:1. In some embodiments, the crystalline composition comprises Compound 1 and an acid in a molar ratio of 1:1.

As used herein, “crystalline,” “crystalline form,” “polymorph,” “Form,” and “form” may be used interchangeably herein, and are meant to include all crystalline and amorphous forms of the compound, including, for example, co-crystals, polymorphs, pseudopolymorphs, salts, solvates, hydrates, unsolvated polymorphs (including anhydrates), conformational polymorphs, and amorphous forms, as well as mixtures thereof, unless a particular crystalline or amorphous form is referred to. Compounds of the present disclosure include crystalline and amorphous forms of those compounds, including, for example, polymorphs, co-crystals, pseudopolymorphs, solvates, hydrates, unsolvated polymorphs (including anhydrates), conformational polymorphs, and amorphous forms of the compounds, as well as mixtures thereof.

As used herein, “co-crystal” may be used to describe crystalline compositions comprising multi-component crystals based on hydrogen bonding interactions of hydrogen ions to form salt forms. In some embodiments, the crystalline composition as described herein are co-crystals comprising an acid (e.g., a weak acid (e.g., oxalic acid)) and Compound 1.

### Definitions

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative, such as those known in the art, for example, described in Remington's Pharmaceutical Sciences 16th edition, Osol, A. Ed. (1980).

As used herein, “treating” and like terms refer to reducing the severity and/or frequency of symptoms, eliminating symptoms and/or the underlying cause of said symptoms, reducing the frequency or likelihood of symptoms and/or their underlying cause, delaying, preventing and/or slowing the progression of diseases and/or disorders and improving or remediating damage caused, directly or indirectly, by the diseases and/or disorders.

As used herein, “delaying development of a disease” or the like, means to defer, hinder, slow, retard, stabilize, and/or postpone development of the disease (such as cancer) or a sign or symptom of the disease. This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the onset of the disease, delaying development of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent and preferably stop) cancer cell infiltration into peripheral organs; inhibit (i.e., slow to some extent and preferably stop) tumor metastasis; inhibiting, to some extent, tumor growth; and/or relieving to some extent one or more of the symptoms associated with the disorder. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. Inhibition may refer to reduction of a disease or symptoms of disease. Inhibition may refer to a reduction in the activity of a particular protein or nucleic acid target. The protein may be deoxycytidine kinase. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “modulator” refers to a composition that increases or decreases the level of a target molecule or the function of a target molecule or the physical state of the target of the molecule.

The term “modulate” is used in accordance with its plain ordinary meaning and refers to the act of changing or varying one or more properties. “Modulation” refers to the process of changing or varying one or more properties. For example, a modulator of a target protein changes by increasing or decreasing a property or function of the target molecule or the amount of the target molecule. A modulator of a disease decreases a symptom, cause, or characteristic of the targeted disease.

“Selective” or “selectivity” or the like of a compound refers to the compound's ability to discriminate between molecular targets.

“Specific”, “specifically”, “specificity”, or the like of a compound refers to the compound's ability to cause a particular action, such as inhibition, to a particular molecular target with minimal or no action to other proteins in the cell.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a compound described herein is administered at the same time, just prior to, or just after the administration of one or more additional therapies, for example, an anticancer agent as described herein. The compounds described herein can be administered alone or can be co-administered to the patient. Co-administration is meant to include simultaneous or sequential administration of the compound individually or in combination (more than one compound or agent). Thus, the preparations can also be combined, when desired, with other active substances (e.g. anticancer agents).

Co-administration includes administering one active agent (e.g. Compound 1 described herein) within 0.5, 1, 2, 4, 6, 8, 10, 12, 16, 20, or 24 hours of a second active agent (e.g. anti-cancer agents). Also contemplated herein, are embodiments, where co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. Co-administration can be accomplished by co-formulation, i.e., preparing a single pharmaceutical composition including both active agents. In other embodiments, the active agents can be formulated separately. In some embodiments, the active and/or adjunctive agents are linked or conjugated to one another. In some embodiments, the compounds described herein are combined with treatments for cancer such as chemotherapy or radiation therapy.

The term “associated” or “associated with” in the context of a substance or substance activity or function associated with a disease means that the disease is caused by (in whole or in part), a symptom of the disease is caused by (in whole or in part) the substance or substance activity or function, or a side-effect of the compound (e.g. toxicity) is caused by (in whole or in part) the substance or substance activity or function.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. A “cancer-patient” is a patient suffering from, or prone to developing cancer.

Unless clearly indicated otherwise, the term “individual” as used herein refers to a mammal, including but not limited to, bovine, horse, feline, rabbit, canine, rodent, or primate (e.g., human). In some embodiments, an individual is a human. In some embodiments, an individual is a non-human primate such as chimpanzees and other apes and monkey species. In some embodiments, an individual is a farm animal such as cattle, horses, sheep, goats and swine; pets such as rabbits, dogs and cats; laboratory animals including rodents, such as rats, mice, and guinea pigs; and the like. In some embodiments, the invention find use in both human medicine and in the veterinary context.

“Disease” or “condition” refer to a state of being or health status of a patient or subject capable of being treated with the compounds or methods provided herein. In some embodiments, the disease as used herein refers to cancer.

As used herein and in the appended claims, the singular forms “a,” “an,” and “the” include plural reference unless the context clearly indicates otherwise.

The term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of 0.5% or less, or variations of 0.1% or less from the specified value.

It is understood that aspect and variations of the invention described herein include “consisting” and/or “consisting essentially of” aspects and variations.

### Crystalline Composition Comprising Compound I (e.g., Comprising Formula I)

The crystalline composition prepared according to the methods of the present disclosure may be characterized by any methodology comprising the art. For example, the crystalline composition comprising Compound 1 ((e.g., comprising Formula I) e.g., prepared comprising the methods of the present disclosure)) may be characterized by X-ray powder diffraction (XRPD), differential scanning calorimetry (DSC), thermogravimetric analysis (TGA), and/or spectroscopy (e.g., Raman, solid state nuclear magnetic resonance (ssNMR), proton nuclear magnetic resonance (¹H NMR), and infrared (IR)). In some embodiments, crystallinity of the solid form is determined by X-Ray Powder Diffraction (XPRD).

Formula 1 may represent a composition comprising two molecules of Compound 1 and one molecules of oxalic acid. As such, Formula I may represent a molar ratio of Compound 1 to oxalic acid in a ratio of about 2:1.

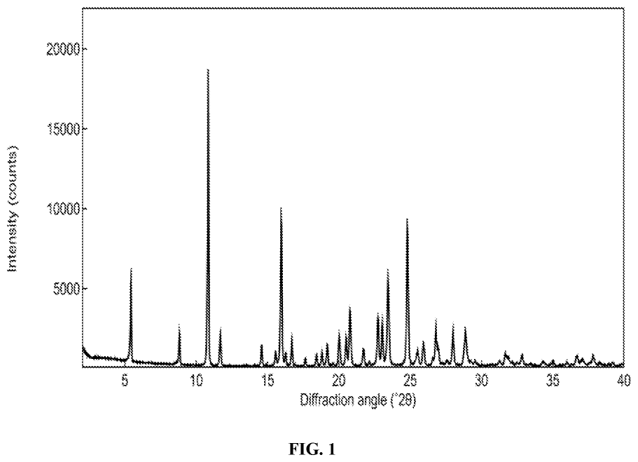

XRPD: Crystalline composition according to the present disclosure may be characterized by XRPD (e.g., crystalline composition(s) comprising the present disclosure can also be identified by its characteristic XRPD peaks, such as show in FIG. 1). The relative intensities of XRPD peaks may vary, depending upon the particle size, the sample preparation technique, the sample mounting procedure and the particular instrument employed. Moreover, instrument variation and other factors may affect the 2-0 values. Therefore, the XRPD peak assignments may vary, for example by plus or minus about 0.2 degrees.

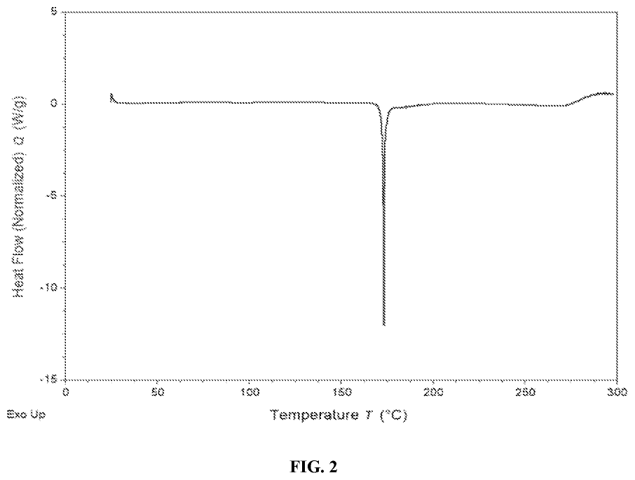

DSC: Crystalline composition according to the present disclosure can also be identified by its characteristic DSC thermograms such as shown in FIG. 2. For DSC, it is known that the temperatures observed may depend upon the rate of temperature change as well as sample preparation technique and the particular instrument employed. Thus, the values reported herein relating to DSC thermograms may vary, for example by plus or minus about 4° C.

TGA: Crystalline composition according to the present disclosure may also give rise to thermal behavior different from that of the amorphous material or other crystalline forms. Thermal behavior may be measured in the laboratory by thermogravimetric analysis (TGA) which may be used to distinguish some crystalline forms from others. In one aspect, the crystalline composition as described herein may be characterized by thermogravimetric analysis.

DVS: Crystalline composition comprising the present disclosure may also give rise to vapors sorption behavior different from that of the amorphous material or other crystalline forms. The vapor sorption behavior may be measured in the laboratory by dynamic vapor sorption (DVS) which may be used to distinguish some crystalline forms from others. In one aspect, the crystalline composition as described herein may be characterized by dynamic vapor sorption.

The crystalline composition comprising Compound 1 (e.g. comprising Formula I) may be useful in the production of medicinal preparations and can be obtained by means of a crystallization process to produce crystalline and semi-crystalline forms or a solidification process to obtain the amorphous form. In some embodiments, the crystallization is carried out by either generating the desired compound (for example Compound 1) in a reaction mixture and isolating the desired crystalline composition from the reaction mixture, or by dissolving raw compound (e.g., Compound 1) in a solvent, optionally with heat, followed by crystallizing/solidifying the product by cooling (including active cooling) and/or by the addition of an antisolvent for a period of time. The crystallization or solidification may be followed by drying carried out under controlled conditions until the desired water content is reached in the end crystalline composition.

FIG. 1 shows the X-ray powder diffraction (XRPD) pattern for the crystalline composition comprising Compound 1 (e.g., comprising Formula I).

FIG. 2 shows the differential scanning calorimetry (DSC) thermogram for the crystalline composition comprising Compound 1 (e.g., comprising Formula I).

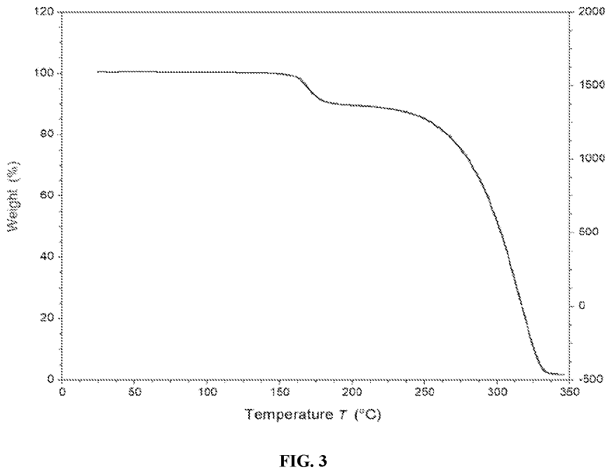

FIG. 3 shows the thermogravimetric analysis (TGA) thermogram for the crystalline composition comprising Compound 1 (e.g., comprising Formula I).

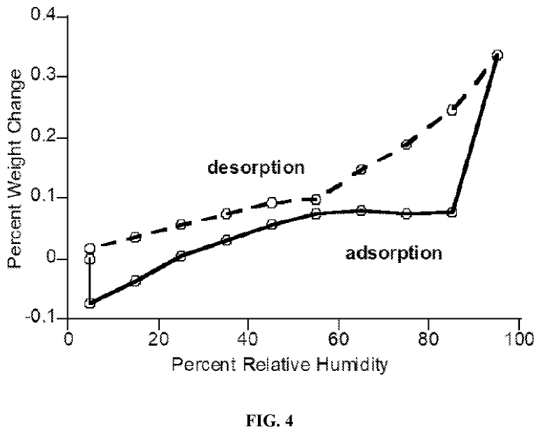

FIG. 4 shows the dynamic vapor sorption (DVS) trace for the crystalline composition comprising Compound 1 (e.g., comprising Formula I).

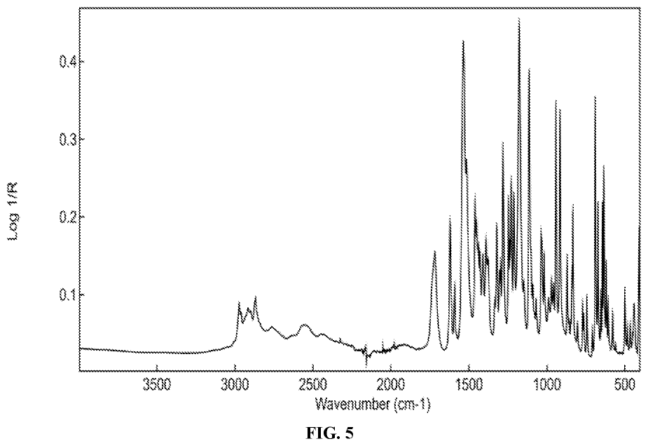

FIG. 5 shows the infrared (IR) spectrum for the crystalline composition comprising Compound 1 (e.g., comprising Formula I).

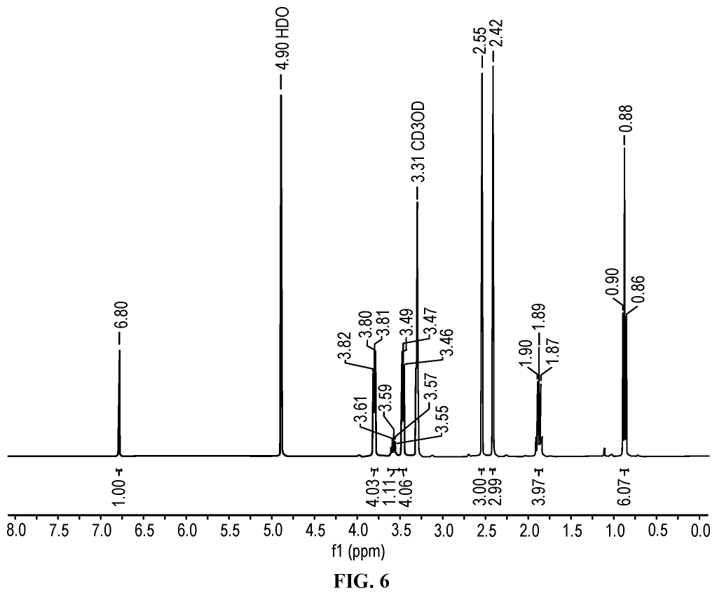

FIG. 6 shows the proton nuclear magnetic resonance (¹H NMR) spectrum for the crystalline composition comprising Compound 1 (e.g., comprising Formula I).

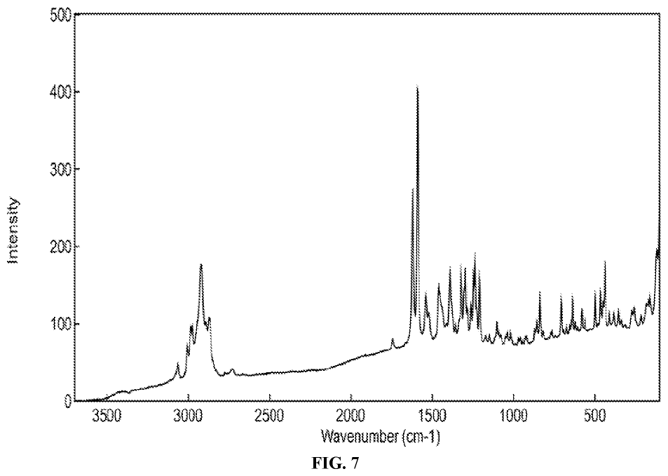

FIG. 7 shows the Raman spectrum for the crystalline composition comprising Compound 1 (e.g., comprising Formula I).

In some aspects, the present disclosure provides a crystalline composition comprising Compound 1. In some embodiments the crystalline composition comprising Compound 1 is a crystalline composition comprising Formula I.

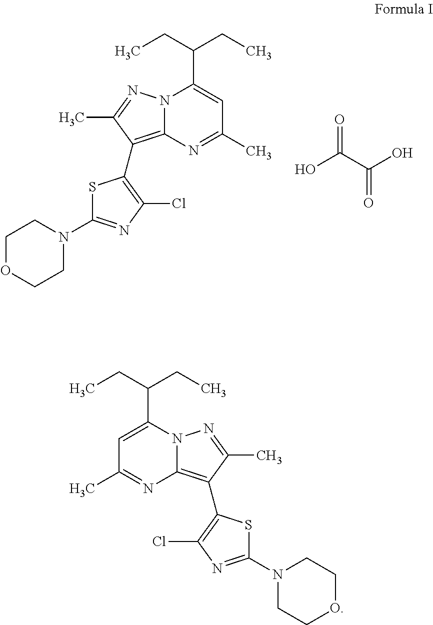

In some embodiments, Formula I represents Compound 1 and oxalic acid in a molar ratio of 2:1.

In some embodiments, the crystals of the crystalline composition comprising Compound 1 (e.g., comprising Formula I) has unit cell parameters at T=150° K of: a=34.003(4) Å, b=6.5843(13) Å, c=21.062(5) Å; β=108.703(12)°, V=4466.5(15) Å³ and a monoclinic C2/c space group.

In some embodiments, the crystals of a composition (e.g., a crystalline composition comprising Compound 1 (e.g., comprising Formula I)) is characterized by:
(a) an X-ray powder diffraction pattern comprising peaks at 10.84±0.2° 2-θ, 15.96±0.2° 2-θ, 23.44±0.2° 2-θ, and 24.80±0.2° 2-θ, and as measured by X-ray powder diffraction using an X-ray wavelength of 0.71073 Å;
(b) an X-ray powder diffraction pattern substantially the same as shown in FIG. 1; (c) a differential scanning calorimetry (DSC) thermogram comprising an endotherm in the range of about 160° C. to 170° C.;
(d) a differential scanning calorimetry (DSC) thermogram comprising an endotherm with an onset of about 164° C. and a peak of about 166° C.;
(e) a differential scanning calorimetry (DSC) thermogram substantially the same as shown in FIG. 2;
(f) a thermogravimetric analysis (TGA) thermogram comprising a loss in mass of about 11% over a temperature range of about 25° C. to about 200° C.;
(g) a thermogravimetric analysis (TGA) thermogram substantially the same as shown in FIG. 3; or
(h) combinations thereof.

In some embodiments, the composition (e.g., crystalline composition comprising a CRF1 antagonist (e.g., Compound 1 (e.g., v Formula I)) is characterized by an X-ray powder diffraction pattern comprising any one or more peaks at 10.84±0.2° 2-θ, 15.96±0.2° 2-θ, 23.44±0.2° 2-θ, and 24.80±0.2° 2-θ, and as measured by X-ray powder diffraction using an X-ray wavelength of 0.71073 Å. In some embodiments, the XRPD pattern comprises at least one of the aforementioned peaks (10.84±0.2° 2-θ, 15.96±0.2° 2-θ, 23.44±0.2° 2-θ, and 24.80±0.2° 2-θ, and as measured by X-ray powder diffraction using an X-ray wavelength of 0.71073 Å). In some embodiments, the XRPD pattern comprises at least two of the aforementioned peaks. In some embodiments, the XRPD pattern comprises at least three of the aforementioned peaks. In some embodiments, the XRPD pattern comprises at least four of the aforementioned peaks. In some embodiments, the composition (e.g., crystalline composition comprising a CRF1 antagonist (e.g., Compound 1 (e.g., comprising Formula I) is characterized by an X-ray powder diffraction pattern comprising peaks at 10.84±0.10 2-θ, 15.96±0.10 2-θ, 23.44±0.10 2-θ, and 24.80±0.10 2-θ, and as measured by X-ray powder diffraction using an X-ray wavelength of 0.71073 Å. In some embodiments, the composition (e.g., crystalline composition comprising a CRF1 antagonist (e.g., Compound 1 (e.g., comprising Formula I) is characterized by an X-ray powder diffraction pattern comprising peaks at about 10.84° 2-θ, about 15.96° 2-θ, about 23.44° 2-θ, and about 24.80° 2-θ, and as measured by X-ray powder diffraction using an X-ray wavelength of 0.71073 Å.

In some embodiments, the X-ray powder diffraction (XRPD) pattern further comprising at least one peak selected from 5.44±0.2° 2-θ, 20.78±0.2° 2-θ, 22.74±0.2° 2-θ, 23.04±0.2° 2-θ, 26.80±0.2° 2-θ, and 28.86±0.2° 2-θ, and as measured by X-ray powder diffraction using an X-ray wavelength of 0.71073 Å. In some embodiments, the X-ray powder diffraction pattern further comprising at least one peak selected from 5.44±0.1° 2-θ, 20.78±0.1° 2-θ, 22.74±0.1° 2-θ, 23.04±0.1° 2-θ, 26.80±0.1° 2-θ, and 28.86±0.1° 2-θ, and as measured by X-ray powder diffraction using an X-ray wavelength of 0.71073 Å. In some embodiments, the X-ray powder diffraction pattern further comprising at least one peak selected from about 5.44° 2-θ, about 20.78° 2-θ, about 22.74° 2-θ, about 23.04° 2-θ, about 26.80° 2-θ, and about 28.86° 2-θ, and as measured by X-ray powder diffraction using an X-ray wavelength of 0.71073 Å.

In some embodiments, the X-ray powder diffraction pattern further comprising at least one peak selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the X-ray powder diffraction pattern further comprising at least two peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-0, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the X-ray powder diffraction pattern further comprising at least three peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the X-ray powder diffraction pattern further comprising at least four peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the X-ray powder diffraction pattern further comprising at least five peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the X-ray powder diffraction pattern further comprising at least six peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the X-ray powder diffraction pattern further comprising at least seven peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the X-ray powder diffraction pattern further comprising at least eight peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å. In some embodiments, the X-ray powder diffraction pattern comprises peaks selected from 8.82±0.1° 2-θ, 11.70±0.1° 2-θ, 14.60±0.1° 2-θ, 15.56±0.1° 2-θ, 16.70±0.1° 2-θ, 18.82±0.1° 2-θ, 19.18±0.1° 2-θ, 20.02±0.1° 2-θ, 20.50±0.1° 2-θ, 21.72±0.1° 2-θ, 25.52±0.1° 2-θ, 25.92±0.1° 2-θ, 26.94±0.1° 2-θ, and 28.00±0.1° 2-θ, and as measured by X-ray powder diffraction using an X-ray wavelength of 0.71073 Å. In some embodiments, the X-ray powder diffraction pattern comprises peaks selected from 8.82° 2-θ, 11.70° 2-θ, 14.60° 2-θ, 15.56° 2-θ, 16.70° 2-θ, 18.82° 2-θ, 19.18° 2-θ, 20.02° 2-θ, 20.50° 2-θ, 21.72° 2-θ, 25.52° 2-θ, 25.92° 2-θ, 26.94° 2-θ, and 28.00° 2-θ, and as measured by X-ray powder diffraction using an X-ray wavelength of 0.71073 Å.

In some embodiments, the composition (e.g., crystalline composition comprising a CRF1 antagonist (e.g., Compound 1 (e.g., comprising Formula I)) is characterized by a differential scanning calorimetry (DSC) thermogram comprising an endotherm in the range of about 160° C. to 170° C. In some embodiments, the crystalline composition is characterized by a differential scanning calorimetry (DSC) thermogram comprising an endotherm with an onset of about 164° C. and a peak of about 166° C. In some embodiments, the crystalline composition is characterized by a melting point of about 166° C. In some embodiments, the crystalline composition is characterized by a differential scanning calorimetry (DSC) thermogram substantially the same as shown in FIG. 2.

In some embodiments, the composition (e.g., crystalline composition comprising a CRF1 antagonist (e.g., Compound 1 (e.g., comprising Formula I))) is characterized by a differential scanning calorimetry (DSC) thermogram comprising an endotherm at about 160° C. to about 170° C., about 160° C. to about 169° C., about 160° C. to about 168° C., about 160° C. to about 167° C., about 160° C. to about 166° C., about 160° C. to about 165° C., about 160° C. to about 164° C., about 160° C. to about 163° C., about 160° C. to about 162° C., about 160° C. to about 161° C., about 161° C. to about 170° C., about 162° C. to about 170° C., about 163° C. to about 170° C., about 164° C. to about 170° C., about 165° C. to about 170° C., about 166° C. to about 170° C., about 167° C. to about 170° C., about 168° C. to about 170° C., or about 169° C. to about 170° C. In some embodiments, the composition (e.g., crystalline composition comprising a CRF1 antagonist (e.g., Compound 1 (e.g., comprising Formula I))) is characterized by a differential scanning calorimetry (DSC) thermogram comprising an endotherm at about 162° C. to about 166° C., for example at about 162° C., about 163° C., about 164° C., 165° C., or 166° C. In some embodiments, the composition (e.g., crystalline composition comprising a CRF1 antagonist (e.g., (e.g., comprising Compound 1))) is characterized by a differential scanning calorimetry (DSC) thermogram comprising an endotherm at about 164° C. In some embodiments, the crystalline composition according for Formula I has a melting point of about 166° C.

In some embodiments, the composition (e.g., crystalline composition comprising a CRF1 antagonist (e.g., Compound 1 (e.g., comprising Formula I))) is characterized by a thermogravimetric analysis (TGA) thermogram comprising a loss in mass of about 11% over a temperature range of about 25° C. to about 200° C. In some embodiments, the crystalline composition decomposes above a temperature of about 50° C., about 100° C., about 150° C., about 200° C., about 250° C., about 300° C., about 350° C., or about 400° C. In some embodiments, the crystalline composition decomposes above a temperature of about 200° C. In some embodiments, the crystalline composition decomposes above a temperature of about 250° C. In some embodiments, the crystalline composition is characterized by a thermogravimetric analysis (TGA) thermogram substantially the same as shown in FIG. 3.

In some embodiments, the composition (e.g., crystalline composition comprising a CRF1 antagonist (e.g., Compound 1 (e.g., comprising Formula I))) is characterized by a dynamic vapor sorption (DVS) trace substantially the same as shown in FIG. 4.

In some embodiments, the composition (e.g., crystalline composition comprising a CRF1 antagonist (e.g., Compound 1 (e.g., comprising Formula I))) is anhydrous.

In some embodiments, the crystalline composition comprising Compound 1 (e.g., comprising Formula I) is stable at room temperature. In some examples, the crystalline composition comprising Compound 1 (e.g., comprising Formula I) can be stored at room temperature for an extended period of time without significant chemical degradation or change in the crystalline form. In some examples, crystalline composition comprising Compound 1 (e.g., comprising Formula I) can be stored at room temperature for a timer period of at least about 10 days, 30 days, 60 days, 90 days, 120 days, 150 days, or 180 days. In some examples, the crystalline composition can be stored at room temperature for a time period of more than about 180 days. In some examples, the crystalline composition can be stored at room temperature for a time period of 10-14 days, 10-18 days, 10-22 days, 10-26 days, 10-30 days, 10-40 days, 10-50 days, 10-60 days, 10-90 days, 10-120 days, 10-150 days, 10-180 days, 14-18 days, 14-22 days, 14-26 days, 14-30 days, 14-40 days, 14-50 days, 14-60 days, 14-90 days, 14-120 days, 14-150 days, 14-180 days, 18-22 days, 18-26 days, 18-30 days, 18-40 days, 18-50 days, 18-60 days, 18-90 days, 18-120 days, 18-150 days, 18-180 days, 22-26 days, 22-30 days, 22-40 days, 22-50 days, 22-60 days, 22-90 days, 22-120 days, 22-150 days, 22-180 days, 26-30 days, 26-40 days, 26-50 days, 26-60 days, 26-90 days, 26-120 days, 26-150 days, 26-180 days, 30-40 days, 30-50 days, 30-60 days, 30-90 days, 30-120 days, 30-150 days, 30-180 days, 40-50 days, 40-60 days, 40-90 days, 40-120 days, 40-150 days, 40-180 days, 50-60 days, 50-90 days, 50-120 days, 50-150 days, 50-180 days, 60-90 days, 60-120 days, 60-150 days, 60-180 days, 90-120 days, 90-150 days, or 90-180 days. In some examples, the crystalline composition can be stored at room temperature for a time period of at least 10 days, 14 days, 18 days, 22 days, 26 days, 30 days, 40 days, 50 days, 60 days, 90 days, 120 days, 150 days, or 180 days.

In some embodiments, the crystalline composition comprising a CRF1 antagonist (e.g., Compound 1 (e.g., comprising Formula I)) is stable at room temperature or temperatures above the room temperature and/or at high relative humidity (RH) (e.g., accelerated aging conditions or long-term storage conditions). In some embodiments, the crystalline composition is stable for at least one month, at least two months, at least three months, at least four months, at least five months, at least six months, at least seven months, at least eight months, at least nine months, at least ten months, at least eleven months, at least twelve months, at least fourteen months, at least sixteen months, and at least eighteen months. In some examples, the crystalline composition can be stored at about 25° C. and at about 75% RH for an extended period of time without significant chemical degradation or change in the crystalline form. In some examples, the crystalline composition comprising a CRF1 antagonist (e.g., Compound 1 (e.g., comprising Formula I)) can be stored at about 25° C. and at about 75% RH for a time period of at least about 10 days, 30 days, 60 days, 90 days, 120 days, 150 days, or 180 days. In some examples, the crystalline composition can be stored at about 25° C. and at about 75% RH for a time period of 10-14 days, 10-18 days, 10-22 days, 10-26 days, 10-30 days, 10-40 days, 10-50 days, 10-60 days, 10-90 days, 10-120 days, 10-150 days, 10-180 days, 14-18 days, 14-22 days, 14-26 days, 14-30 days, 14-40 days, 14-50 days, 14-60 days, 14-90 days, 14-120 days, 14-150 days, 14-180 days, 18-22 days, 18-26 days, 18-30 days, 18-40 days, 18-50 days, 18-60 days, 18-90 days, 18-120 days, 18-150 days, 18-180 days, 22-26 days, 22-30 days, 22-40 days, 22-50 days, 22-60 days, 22-90 days, 22-120 days, 22-150 days, 22-180 days, 26-30 days, 26-40 days, 26-50 days, 26-60 days, 26-90 days, 26-120 days, 26-150 days, 26-180 days, 30-40 days, 30-50 days, 30-60 days, 30-90 days, 30-120 days, 30-150 days, 30-180 days, 40-50 days, 40-60 days, 40-90 days, 40-120 days, 40-150 days, 40-180 days, 50-60 days, 50-90 days, 50-120 days, 50-150 days, 50-180 days, 60-90 days, 60-120 days, 60-150 days, 60-180 days, 90-120 days, 90-150 days, or 90-180 days. In some examples, the crystalline composition comprising Compound 1 (e.g., comprising Formula I) can be stored at about 25° C. for a time period of at least 10 days, 14 days, 18 days, 22 days, 26 days, 30 days, 40 days, 50 days, 60 days, 90 days, 120 days, 150 days, or 180 days.

In some embodiments, the crystalline composition comprising Compound 1 (e.g., comprising Formula I) is thermodynamically stable.

In some embodiments, the crystalline composition comprising Compound 1 (e.g., comprising Formula I) is highly bioavailable.

In some embodiments, the crystalline composition comprising Compound 1 (e.g., comprising Formula I) is stable under accelerated storage conditions.

In some embodiments, the crystalline composition comprising Compound 1 (e.g., comprising Formula I) is characterized by an infrared (IR) spectrum substantially the same as shown in FIG. 5.

In some embodiments, the crystalline composition comprising Compound 1 (e.g., comprising Formula I) is characterized by a proton nuclear magnetic resonance (¹H NMR) spectrum substantially the same as shown in FIG. 6.

In some embodiments, the crystalline composition comprising Compound 1 (e.g., comprising Formula I) is characterized by a Raman spectrum substantially the same as shown in FIG. 7.

### Compositions and Formulations

In some aspects, the present disclosure provides a pharmaceutical composition comprising a crystalline composition comprising Formula I.

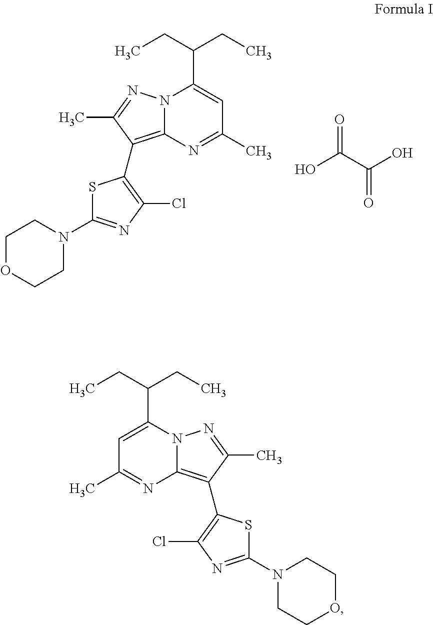

and at least one pharmaceutically acceptable carrier or excipient.

In some aspects, the present disclosure provides a pharmaceutical composition comprising Compound 1:

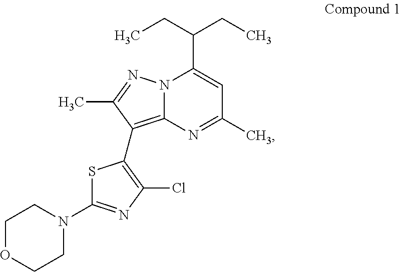 [SMILES: CCC(CC)c1cc(C)nc2c(-c3sc(N4CCOCC4)nc3Cl)c(C)nn12]

or a pharmaceutically acceptable salt thereof, and at least one pharmaceutically acceptable carrier or excipient. In some embodiments, the pharmaceutical composition is a crystalline pharmaceutical composition comprising Compound 1. In some embodiments, the crystalline pharmaceutical composition comprising Compound 1 comprises Formula I.

### Dosage Form

In some embodiments, the pharmaceutical composition described herein is provided in unit dosage form. As used herein, a “unit dosage form” is a composition containing an amount of the crystalline composition comprising Compound 1 (e.g., comprising Formula I) that is suitable for administration to a subject in a single dose. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded.

In some embodiments, the pharmaceutical compositions described herein are formulated as oral dosage forms. Suitable oral dosage forms include, for example, tablets, pills, sachets, or capsules. In some embodiments, the pharmaceutical composition comprises one or more additional pharmaceutically acceptable excipients. See, e.g., Remington: The Science and Practice of Pharmacy (Gennaro, 21st Ed. Mack Pub. Co., Easton, PA (2005) for a list of pharmaceutically acceptable excipients.

### Capsule

In some embodiments, the pharmaceutical composition comprising the crystalline composition comprising Compound 1 (e.g., comprising Formula I) is formulated as a capsule. In some embodiments, the pharmaceutical composition is encapsulated in a hard gel capsule. In some embodiments, the pharmaceutical composition is encapsulated in a soft gel capsule. In some embodiments, the pharmaceutical composition is formulated as a hard gel capsule. In some embodiments, the pharmaceutical composition is formulated as a soft gel capsule.

In some embodiments, the pharmaceutical composition comprising Compound 1 is formulated as a capsule. In some embodiments, the pharmaceutical composition is encapsulated in a hard gel capsule. In some embodiments, the pharmaceutical composition is encapsulated in a soft gel capsule. In some embodiments, the pharmaceutical composition is formulated as a hard gel capsule. In some embodiments, the pharmaceutical composition is formulated as a soft gel capsule.

In some embodiments, the capsule is formed using materials which include, but are not limited to, natural or synthetic gelatin, pectin, casein, collagen, protein, modified starch, polyvinylpyrrolidone, acrylic polymers, cellulose derivatives, or any combinations thereof. In some embodiments, the capsule is formed using preservatives, coloring and opacifying agents, flavorings and sweeteners, sugars, gastroresistant substances, or any combinations thereof. In some embodiments, the capsule is coated. In some embodiments, the coating covering the capsule includes, but is not limited to, immediate release coatings, protective coatings, enteric or delayed release coatings, sustained release coatings, barrier coatings, seal coatings, or combinations thereof. In some embodiments, a capsule herein is hard or soft. In some embodiments, the capsule is seamless. In some embodiments, the capsule is broken such that the particulates are sprinkled on soft foods and swallowed without chewing. In some embodiments, the shape and size of the capsule also vary. Examples of capsule shapes include, but are not limited to, round, oval, tubular, oblong, twist off, or a non-standard shape. The size of the capsule may vary comprising the volume of the particulates. In some embodiments, the size of the capsule is adjusted based on the volume of the particulates and powders. Hard or soft gelatin capsules may be manufactured in accordance with conventional methods as a single body unit comprising the standard capsule shape. A single-body soft gelatin capsule typically may be provided, for example, in sizes from 3 to 22 minims (1 minims being equal to 0.0616 ml) and in shapes of oval, oblong or others. The gelatin capsule may also be manufactured in accordance with conventional methods, for example, as a two-piece hard gelatin capsule, sealed or unsealed, typically in standard shape and various standard sizes, conventionally designated as (000), (00), (0), (1), (2), (3), (4), and (5). The largest number corresponds to the smallest size. In some embodiments, the pharmaceutical composition described herein (e.g., capsule) is swallowed as a whole.

In some embodiments, the capsule comprises one or more pharmaceutically acceptable excipients.

In some embodiments, the capsule is free of additional excipients.

### Tablet

In some embodiments, the pharmaceutical composition comprising the crystalline composition comprising Compound I (e.g., comprising Formula I) is formulated as a tablet.

In some embodiments, the pharmaceutical composition comprising Compound 1 is formulated as a tablet.

In some embodiments, the tablet size is less than about 1000 mg, less than about 800 mg, less than about 600 mg, less than about 400 mg or less than about 200 mg. In some embodiments, the tablet has a dose strength of more than about 50 mg, more than about 100 mg, more than about 150 mg, more than about 200 mg, or more than about 250 mg. In some embodiments, the tablet size is less than about 1000 mg for a dose strength of more than about 50 mg. In some embodiments, the tablet size is less than 800 mg for a dose strength of more than about 100 mg. In some embodiments, the tablet size is less than 600 mg for a dose strength of more than about 150 mg. In some embodiment, the tablet size is less than 400 mg for a dose strength of more than about 200 mg. In some embodiments, the tablet size is less than 400 mg for a dose strength of 200 mg. In some embodiments, the tablet size is about 200 mg for a dose strength of about 50 mg.

In some embodiments, more than about 20% of the tablet is dissolved in conventional dissolution media. In some embodiments, more than about 40% of the tablet is dissolved in conventional dissolution media. In some embodiments, more than about 50% of the tablet is dissolved in conventional dissolution media. In some embodiments, more than about 60% of the tablet is dissolved in conventional dissolution media. In some embodiments, more than about 70% of the tablet is dissolved in conventional dissolution media. In some embodiments, more than about 80% of the tablet is dissolved in conventional dissolution media. In some embodiments, more than about 20% of the tablet is dissolved in less than 24 hours in conventional dissolution media. In some embodiments, more than about 20% of the tablet is dissolved in less than 12 hours in conventional dissolution media. In some embodiments, more than about 20% of the tablet is dissolved in less than 6 hours in conventional dissolution media. In some embodiments, more than about 20% of the tablet is dissolved in less than 3 hours in conventional dissolution media. In some embodiments, more than about 20% of the tablet is dissolved in less than 2 hours in conventional dissolution media. In some embodiments, more than about 20% of the tablet is dissolved in less than 60 minutes in conventional dissolution media. In some embodiments, more than about 40% of the tablet is dissolved in less than 60 minutes in conventional dissolution media. In some embodiments, more than about 50% of the tablet is dissolved in less than 60 minutes in conventional dissolution media. In some embodiments, more than about 60% of the tablet is dissolved in less than 60 minutes in conventional dissolution media. In some embodiments, more than about 70% of the tablet is dissolved in less than 60 minutes in conventional dissolution media. In some embodiments, more than about 80% of the tablet is dissolved in less than 60 minutes in conventional dissolution media. In some embodiments, more than 70% of the tablet is dissolved in 60 minutes in conventional dissolution media.

In some embodiments, the tablet is produced at a commercial scale.

In some embodiments, the tablet comprises one or more pharmaceutically acceptable excipients.

In some embodiments, the tablet is coated with a coating material, e.g., a sealant. In some embodiments, the coating material is water soluble. In some embodiments, the coating material comprises a polymer, plasticizer, a pigment, or any combination thereof. In some embodiments, the coating material is in the form of a film coating, e.g., a glossy film, a pH independent film coating, an aqueous film coating, a dry powder film coating (e.g., complete dry powder film coating), or any combination thereof. In some embodiments, the coating material is highly adhesive. In some embodiments, the coating material provides low level of water permeation. In some embodiments, the coating material provides oxygen barrier protection. In some embodiments, the coating material allows immediate disintegration for fast release of Compound 1. In some embodiments, the coating material is pigmented, clear, or white. In some embodiments, the coating is an enteric coating. Exemplary coating materials include, without limitation, polyvinylpyrrolidone, polyvinyl alcohol, an acrylate-methacrylic acid copolymer, a methacrylate-methacrylic acid copolymer, cellulose acetate phthalate, cellulose acetate succinate, hydroxypropyl methylcellulose phthalate, hydroxypropyl methylcellulose acetate succinate, polyvinyl acetate phthalate, shellac, cellulose acetate trimellitate, sodium alginate, zein, and any combinations thereof.

### Oral Dosage Forms

In some embodiments, the pharmaceutical composition comprising Compound 1 or the crystalline composition comprising Formula I or is formulated as an oral dosage form. In some embodiments, the oral dosage form is selected from the group consisting of a tablet, a capsule, a buccal tablet, a sub-lingual table, an orally-disintegrating tablet, a thin film, a liquid solution, a liquid suspension, a syrup, a powder, and solid crystals. In some embodiment, the oral dosage form is selected from the group consisting of a tablet, a capsule, a buccal tablet, a sub-lingual table, an orally-disintegrating tablet, a thin film, a liquid solution, a liquid suspension, a syrup, a powder, solid crystals, minitabs, coated pellets and sachets.

Oral dosage forms may include capsules, tablets, pills, powders or granules. Such forms may include forms that dissolve or disintegrate quickly in the oral environment. Such forms may be prepared with coatings and shells. In some embodiments, these oral dosage forms are capable of controlled or sustained release.

### Pharmaceutically Acceptable Excipients

In some embodiments, the pharmaceutical composition comprising Compound 1 (e.g., crystalline composition comprising Compound 1) or the crystalline composition comprising Formula I comprises a pharmaceutically acceptable excipient. In some embodiments, the pharmaceutical composition is free of pharmaceutically acceptable excipients. The term “pharmaceutically acceptable excipient”, as used herein, means one or more compatible solid or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy of the composition under ordinary use situations. In some embodiments, the pharmaceutically acceptable excipient is of sufficiently high purity and sufficiently low toxicity to render them suitable for administration preferably to an animal, preferably mammal, being treated.

Some examples of substances, which can serve as pharmaceutically acceptable excipients include:
Amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, the amino acid is arginine. In some embodiments, the amino acid is L-arginine.
Monosaccharides such as glucose (dextrose), arabinose, mannitol, fructose (levulose), and galactose.
Cellulose and its derivatives such as sodium carboxymethyl cellulose, ethyl cellulose, and methyl cellulose.
Solid lubricants such as talc, stearic acid, magnesium stearate and sodium stearyl fumarate.
Polyols such as propylene glycol, glycerin, sorbitol, mannitol, and polyethylene glycol.
Emulsifiers such as the polysorbates.
Wetting agents such as sodium lauryl sulfate, Tween®, Span, alkyl sulphates, and alkyl ethoxylate sulphates.
Cationic surfactants such as cetrimide, benzalkonium chloride, and cetylpyridinium chloride.
Diluents such as calcium carbonate, microcrystalline cellulose, calcium phosphate, starch, pregelatinized starch, sodium carbonate, mannitol, and lactose.
Binders such as starches (corn starch and potato starch), gelatin, sucrose hydroxypropyl cellulose (HPC), polyvinylpyrrolidone (PVP), and hydroxypropyl methyl cellulose (HPMC).
Disintegrants such as starch, and alginic acid.
Super-disintegrants such as ac-di-sol, croscarmellose sodium, sodium starch glycolate and crospovidone.
Glidants such as silicon dioxide.
Coloring agents such as the FD&C dyes.
Sweeteners and flavoring agents, such as aspartame, saccharin, menthol, peppermint, and fruit flavors.
Preservatives such as benzalkonium chloride, PHMB, chlorobutanol, thimerosal, phenylmercuric, acetate, phenylmercuric nitrate, parabens, and sodium benzoate.
Tonicity adjustors such as sodium chloride, potassium chloride, mannitol, and glycerin.
Antioxidants such as sodium bisulfite, acetone sodium bisulfite, sodium formaldehyde, sulfoxylate, thiourea, and EDTA.
pH adjuster such as NaOH, sodium carbonate, sodium acetate, HCl, and citric acid.
Cryoprotectants such as sodium or potassium phosphates, citric acid, tartaric acid, gelatin, and carbohydrates such as dextrose, mannitol, and dextran.
Surfactants such as sodium lauryl sulfate. For example, cationic surfactants such as cetrimide (including tetradecyl trimethyl ammonium bromide with dodecyl and hexadecyl compounds), benzalkonium chloride, and cetylpyridinium chloride. Some examples of anionic surfactants are alkylsulphates, alkylethoxylate sulphates, soaps, carboxylate ions, sulfate ions, and sulfonate ions. Some examples of non-ionic surfactants are polyoxyethylene derivatives, polyoxypropylene derivatives, polyol derivatives, polyol esters, polyoxyethylene esters, poloxamers, glocol, glycerol esters, sorbitan derivatives, polyethylene glycol (such as PEG-40, PEG-50, or PEG-55) and esters of fatty alcohols.
Organic materials such as carbohydrates, modified carbohydrates, lactose (including a-lactose, monohydrate spray dried lactose or anhydrous lactose), starch, pregelatinized starch, sucrose, mannitol, sorbital, cellulose (including powdered cellulose and microcrystalline cellulose).
Inorganic materials such as calcium phosphates (including anhydrous dibasic calcium phosphate, dibasic calcium phosphate or tribasic calcium phosphate).
Co-processed diluents.
Compression aids.
Anti-tacking agents such as silicon dioxide and talc.

In some embodiments, the pharmaceutical composition comprises the crystalline composition comprising Formula I.

In some embodiments, the pharmaceutical composition (e.g., crystalline composition) comprises Compound 1.

In some embodiments, the pharmaceutical composition comprises a diluent in about 5% to about 95% by weight of the dosage form. In some embodiments, the pharmaceutical composition comprises a diluent in about 5% to about 90% by weight of the dosage form. In some embodiments, the pharmaceutical composition comprises a diluent in about 5% to about 85% by weight of the dosage form. In some embodiments, the pharmaceutical composition comprises a diluent in about 5% to about 80% by weight of the dosage form. In some embodiments, the pharmaceutical composition comprises a diluent in about 5% to about 75% by weight of the dosage form. In some embodiments, the pharmaceutical composition comprises a diluent in about 5% to about 70% by weight of the dosage form. In some embodiments, the pharmaceutical composition comprises a diluent in about 5% to about 65% by weight of the dosage form. In some embodiments, the pharmaceutical composition comprises a diluent in about 5% to about 60% by weight of the dosage form. In some embodiments, the pharmaceutical composition comprises a diluent in about 5% to about 55% by weight of the dosage form. In some embodiments, the pharmaceutical composition comprises a diluent in about 5% to about 50% by weight of the dosage form. In some embodiments, the pharmaceutical composition comprises a diluent in about 5% to about 45% by weight of the dosage form. In some embodiments, the pharmaceutical composition comprises a diluent in about 5% to about 40% by weight of the dosage form. In some embodiments, the pharmaceutical composition comprises a diluent in about 5% to about 35% by weight of the dosage form. In some embodiments, the pharmaceutical composition comprises a diluent in about 5% to about 30% by weight of the dosage form. In some embodiments, the pharmaceutical composition comprises a diluent in about 5% to about 25% by weight of the dosage form. In some embodiments, the pharmaceutical composition comprises a diluent in about 5% to about 20% by weight of the dosage form.

In some embodiments, the pharmaceutical composition comprises a diluent in about 95% by weight of the dosage form. In some embodiments, the pharmaceutical composition comprises a diluent in about 90% by weight of the dosage form. In some embodiments, the pharmaceutical composition comprises a diluent in about 85% by weight of the dosage form. In some embodiments, the pharmaceutical composition comprises a diluent in about 80% by weight of the dosage form. In some embodiments, the pharmaceutical composition comprises a diluent in about 75% by weight of the dosage form. In some embodiments, the pharmaceutical composition comprises a diluent in about 70% by weight of the dosage form. In some embodiments, the pharmaceutical composition comprises a diluent in about 65% by weight of the dosage form. In some embodiments, the pharmaceutical composition comprises a diluent in about 60% by weight of the dosage form. In some embodiments, the pharmaceutical composition comprises a diluent in about 55% by weight of the dosage form. In some embodiments, the pharmaceutical composition comprises a diluent in about 50% by weight of the dosage form. In some embodiments, the pharmaceutical composition comprises a diluent in about 45% by weight of the dosage form. In some embodiments, the pharmaceutical composition comprises a diluent in about 40% by weight of the dosage form. In some embodiments, the pharmaceutical composition comprises a diluent in about 35% by weight of the dosage form. In some embodiments, the pharmaceutical composition comprises a diluent in about 30% by weight of the dosage form. In some embodiments, the pharmaceutical composition comprises a diluent in about 25% by weight of the dosage form. In some embodiments, the pharmaceutical composition comprises a diluent in about 20% by weight of the dosage form. In some embodiments, the pharmaceutical composition comprises a diluent in about 10% by weight of the dosage form. In some embodiments, the pharmaceutical composition comprises a diluent in about 5% by weight of the dosage form.

In some embodiments, the pharmaceutical composition comprises a second diluent. In some embodiments, the pharmaceutical composition comprises a second diluent in about 5% to about 95% by weight of the dosage form. In some embodiments, the pharmaceutical composition comprises a second diluent in about 5% to about 90% by weight of the dosage form. In some embodiments, the pharmaceutical composition comprises a second diluent in about 5% to about 85% by weight of the dosage form. In some embodiments, the pharmaceutical composition comprises a second diluent in about 5% to about 80% by weight of the dosage form. In some embodiments, the pharmaceutical composition comprises a second diluent in about 5% to about 75% by weight of the dosage form. In some embodiments, the pharmaceutical composition comprises a second diluent in about 5% to about 70% by weight of the dosage form. In some embodiments, the pharmaceutical composition comprises a second diluent in about 5% to about 65% by weight of the dosage form. In some embodiments, the pharmaceutical composition comprises a second diluent in about 5% to about 60% by weight of the dosage form. In some embodiments, the pharmaceutical composition comprises a second diluent in about 5% to about 55% by weight of the dosage form. In some embodiments, the pharmaceutical composition comprises a second diluent in about 5% to about 50% by weight of the dosage form. In some embodiments, the pharmaceutical composition comprises a second diluent in about 5% to about 45% by weight of the dosage form. In some embodiments, the pharmaceutical composition comprises a second diluent in about 5% to about 40% by weight of the dosage form. In some embodiments, the pharmaceutical composition comprises a second diluent in about 5% to about 35% by weight of the dosage form. In some embodiments, the pharmaceutical composition comprises a second diluent in about 5% to about 30% by weight of the dosage form. In some embodiments, the pharmaceutical composition comprises a second diluent in about 5% to about 25% by weight of the dosage form. In some embodiments, the pharmaceutical composition comprises a second diluent in about 5% to about 20% by weight of the dosage form.

In some embodiments, the pharmaceutical composition comprises a second diluent in about 95% by weight of the dosage form. In some embodiments, the pharmaceutical composition comprises a second diluent in about 90% by weight of the dosage form. In some embodiments, the pharmaceutical composition comprises a second diluent in about 85% by weight of the dosage form. In some embodiments, the pharmaceutical composition comprises a second diluent in about 80% by weight of the dosage form. In some embodiments, the pharmaceutical composition comprises a second diluent in about 75% by weight of the dosage form. In some embodiments, the pharmaceutical composition comprises a second diluent in about 70% by weight of the dosage form. In some embodiments, the pharmaceutical composition comprises a second diluent in about 65% by weight of the dosage form. In some embodiments, the pharmaceutical composition comprises a second diluent in about 60% by weight of the dosage form. In some embodiments, the pharmaceutical composition comprises a second diluent in about 55% by weight of the dosage form. In some embodiments, the pharmaceutical composition comprises a second diluent in about 50% by weight of the dosage form. In some embodiments, the pharmaceutical composition comprises a second diluent in about 45% by weight of the dosage form. In some embodiments, the pharmaceutical composition comprises a second diluent in about 40% by weight of the dosage form. In some embodiments, the pharmaceutical composition comprises a second diluent in about 35% by weight of the dosage form. In some embodiments, the pharmaceutical composition comprises a second diluent in about 30% by weight of the dosage form. In some embodiments, the pharmaceutical composition comprises a second diluent in about 25% by weight of the dosage form. In some embodiments, the pharmaceutical composition comprises a second diluent in about 20% by weight of the dosage form. In some embodiments, the pharmaceutical composition comprises a second diluent in about 10% by weight of the dosage form. In some embodiments, the pharmaceutical composition comprises a second diluent in about 5% by weight of the dosage form.

In some embodiments, the diluent is selected from calcium carbonate, microcrystalline cellulose, calcium phosphate, starch, pregelatinized starch, sodium carbonate, mannitol, and lactose. In some embodiments, the diluent is lactose monohydrate. In some embodiments, the diluent is microcrystalline cellulose. In some embodiments, the diluent is calcium carbonate. In some embodiments, the diluent is calcium phosphate. In some embodiments, the diluent is starch. In some embodiments, the diluent is pregelatinized starch. In some embodiments, the diluent is sodium carbonate. In some embodiments, the diluent is mannitol.

In some embodiments, the pharmaceutical composition comprises a binder in about 1% to about 10% by weight of the dosage form. In some embodiments, the pharmaceutical composition comprises a binder in about 1% to about 9% by weight of the dosage form. In some embodiments, the pharmaceutical composition comprises a binder in about 1% to about 8% by weight of the dosage form. In some embodiments, the pharmaceutical composition comprises a binder in about 1% to about 7% by weight of the dosage form. In some embodiments, the pharmaceutical composition comprises a binder in about 1% to about 6% by weight of the dosage form. In some embodiments, the pharmaceutical composition comprises a binder in about 1% to about 5% by weight of the dosage form. In some embodiments, the pharmaceutical composition comprises a binder in about 1% to about 4% by weight of the dosage form. In some embodiments, the pharmaceutical composition comprises a binder in about 1% to about 3% by weight of the dosage form. In some embodiments, the pharmaceutical composition comprises a binder in about 1% to about 2% by weight of the dosage form.

In some embodiments, the pharmaceutical composition comprises a binder in about 10% by weight of the dosage form. In some embodiments, the pharmaceutical composition comprises a binder in about 9% by weight of the dosage form. In some embodiments, the pharmaceutical composition comprises a binder in about 8% by weight of the dosage form. In some embodiments, the pharmaceutical composition comprises a binder in about 7% by weight of the dosage form. In some embodiments, the pharmaceutical composition comprises a binder in about 6% by weight of the dosage form. In some embodiments, the pharmaceutical composition comprises a binder in about 5% by weight of the dosage form. In some embodiments, the pharmaceutical composition comprises a binder in about 4% by weight of the dosage form. In some embodiments, the pharmaceutical composition comprises a binder in about 3% by weight of the dosage form. In some embodiments, the pharmaceutical composition comprises a binder in about 2% by weight of the dosage form. In some embodiments, the pharmaceutical composition comprises a binder in about 1% by weight of the dosage form.

In some embodiments, the binder is selected from starches (corn starch and potato starch), gelatin, sucrose hydroxypropyl cellulose (HPC), polyvinylpyrrolidone (PVP), and hydroxypropyl methyl cellulose (HPMC). In some embodiments, the binder is hydroxypropyl cellulose (HPC). In some embodiments, the binder is starch. In some embodiments, the binder is gelatin. In some embodiments, the binder is polyvinylpyrrolidone (PVP). In some embodiments, the binder is hydroxypropyl methyl cellulose (HPMC).

In some embodiments, the pharmaceutical composition comprises a surfactant in about 0.5% to about 5% by weight of the dosage form. In some embodiments, the pharmaceutical composition comprises a surfactant in about 0.5% to about 4.5% by weight of the dosage form. In some embodiments, the pharmaceutical composition comprises a surfactant in about 0.5% to about 4.0% by weight of the dosage form. In some embodiments, the pharmaceutical composition comprises a surfactant in about 0.5% to about 3.5% by weight of the dosage form. In some embodiments, the pharmaceutical composition comprises a surfactant in about 0.5% to about 3.0% by weight of the dosage form. In some embodiments, the pharmaceutical composition comprises a surfactant in about 0.5% to about 2.5% by weight of the dosage form. In some embodiments, the pharmaceutical composition comprises a surfactant in about 0.5% to about 2.0% by weight of the dosage form. In some embodiments, the pharmaceutical composition comprises a surfactant in about 0.5% to about 1.5% by weight of the dosage form. In some embodiments, the pharmaceutical composition comprises a surfactant in about 0.5% to about 1% by weight of the dosage form. In some embodiments, the pharmaceutical composition comprises a surfactant in about 0.5% to about 0.8% by weight of the dosage form.

In some embodiments, the pharmaceutical composition comprises a surfactant in about 5% by weight of the dosage form. In some embodiments, the pharmaceutical composition comprises a surfactant in about 4.5% by weight of the dosage form. In some embodiments, the pharmaceutical composition comprises a surfactant in about 4.0% by weight of the dosage form. In some embodiments, the pharmaceutical composition comprises a surfactant in about 3.5% by weight of the dosage form. In some embodiments, the pharmaceutical composition comprises a surfactant in about 3.0% by weight of the dosage form. In some embodiments, the pharmaceutical composition comprises a surfactant in about 2.5% by weight of the dosage form. In some embodiments, the pharmaceutical composition comprises a surfactant in about 2.0% by weight of the dosage form. In some embodiments, the pharmaceutical composition comprises a surfactant in about 1.5% by weight of the dosage form. In some embodiments, the pharmaceutical composition comprises a surfactant in about 1% by weight of the dosage form. In some embodiments, the pharmaceutical composition comprises a surfactant in about 0.75% by weight of the dosage form. In some embodiments, the pharmaceutical composition comprises a surfactant in about 0.50% by weight of the dosage form. In some embodiments, the pharmaceutical composition comprises a surfactant in about 0.25% by weight of the dosage form. In some embodiments, the pharmaceutical composition comprises a surfactant in about 0.1% by weight of the dosage form.

In some embodiments, the surfactant is selected from sodium lauryl sulfate, cetrimide, benzalkonium chloride, cetylpyridinium chloride, alkylsulphates, alkylethoxylate sulphates, soaps, carboxylate ions, sulfate ions, sulfonate ions, polyoxyethylene derivatives, polyoxypropylene derivatives, polyol derivatives, polyol esters, polyoxyethylene esters, poloxamers, glocol, glycerol esters, sorbitan derivatives, polyethylene glycol (such as PEG-40, PEG-50, or PEG-55) and esters of fatty alcohols. In some embodiments, the surfactant is sodium lauryl sulfate.

In some embodiments, the pharmaceutical composition comprises a disintegrant in about 1% to about 10% by weight of the dosage form. In some embodiments, the pharmaceutical composition comprises a disintegrant in about 1% to about 9% by weight of the dosage form. In some embodiments, the pharmaceutical composition comprises a disintegrant in about 1% to about 8% by weight of the dosage form. In some embodiments, the pharmaceutical composition comprises a disintegrant in about 1% to about 7% by weight of the dosage form. In some embodiments, the pharmaceutical composition comprises a disintegrant in about 1% to about 6% by weight of the dosage form. In some embodiments, the pharmaceutical composition comprises a disintegrant in about 1% to about 5% by weight of the dosage form. In some embodiments, the pharmaceutical composition comprises a disintegrant in about 1% to about 4% by weight of the dosage form. In some embodiments, the pharmaceutical composition comprises a disintegrant in about 1% to about 3% by weight of the dosage form. In some embodiments, the pharmaceutical composition comprises a disintegrant in about 1% to about 2% by weight of the dosage form. In some embodiments, the pharmaceutical composition comprises a disintegrant in about 1% to about 1.5% by weight of the dosage form.

In some embodiments, the pharmaceutical composition comprises a disintegrant in about 10% by weight of the dosage form. In some embodiments, the pharmaceutical composition comprises a disintegrant in about 9% by weight of the dosage form. In some embodiments, the pharmaceutical composition comprises a disintegrant in about 8% by weight of the dosage form. In some embodiments, the pharmaceutical composition comprises a disintegrant in about 7% by weight of the dosage form. In some embodiments, the pharmaceutical composition comprises a disintegrant in about 6% by weight of the dosage form. In some embodiments, the pharmaceutical composition comprises a disintegrant in about 5% by weight of the dosage form. In some embodiments, the pharmaceutical composition comprises a disintegrant in about 4% by weight of the dosage form. In some embodiments, the pharmaceutical composition comprises a disintegrant in about 3% by weight of the dosage form. In some embodiments, the pharmaceutical composition comprises a disintegrant in about 2% by weight of the dosage form. In some embodiments, the pharmaceutical composition comprises a disintegrant in about 1% by weight of the dosage form.

In some embodiments, the disintegrant is selected from starch, alginic acid, ac-di-sol, croscarmellose sodium, sodium starch glycolate, and crospovidone. In some embodiments the disintegrant is croscarmellose sodium. In some embodiments the disintegrant is sodium starch glycolate. In some embodiments the disintegrant is crospovidone.

In some embodiments, the pharmaceutical composition comprises a glidant in about 1% to about 5% by weight of the dosage form. In some embodiments, the pharmaceutical composition comprises a glidant in about 1% to about 4% by weight of the dosage form. In some embodiments, the pharmaceutical composition comprises a glidant in about 1% to about 3% by weight of the dosage form. In some embodiments, the pharmaceutical composition comprises a glidant in about 1% to about 2% by weight of the dosage form.

In some embodiments, the pharmaceutical composition comprises a glidant in about 5% by weight of the dosage form. In some embodiments, the pharmaceutical composition comprises a glidant in about 4% by weight of the dosage form. In some embodiments, the pharmaceutical composition comprises a glidant in about 3% by weight of the dosage form. In some embodiments, the pharmaceutical composition comprises a glidant in about 2% by weight of the dosage form. In some embodiments, the pharmaceutical composition comprises a glidant in about 1% by weight of the dosage form.

In some embodiments the glidant is colloidal silicon dioxide.

In some embodiments, the pharmaceutical composition comprises a lubricant in about 1% to about 5% by weight of the dosage form. In some embodiments, the pharmaceutical composition comprises a lubricant in about 1% to about 4% by weight of the dosage form. In some embodiments, the pharmaceutical composition comprises a lubricant in about 1% to about 3% by weight of the dosage form. In some embodiments, the pharmaceutical composition comprises a lubricant in about 1% to about 2% by weight of the dosage form.

In some embodiments, the pharmaceutical composition comprises a lubricant in about 5% by weight of the dosage form. In some embodiments, the pharmaceutical composition comprises a lubricant in about 4% by weight of the dosage form. In some embodiments, the pharmaceutical composition comprises a lubricant in about 3% by weight of the dosage form. In some embodiments, the pharmaceutical composition comprises a lubricant in about 2% by weight of the dosage form. In some embodiments, the pharmaceutical composition comprises a lubricant in about 1% by weight of the dosage form.

In some embodiments the lubricant is selected from talc, stearic acid, magnesium stearate and sodium stearyl fumarate. In some embodiments, the lubricant is talc. In some embodiments, the lubricant is stearic acid. In some embodiments, the lubricant is magnesium stearate. In some embodiments, the lubricant is sodium stearyl fumarate.

In some embodiments, the pharmaceutical composition comprises a granulating agent. In some embodiments, the granulating agent is purified water.

In some embodiments, the pharmaceutical composition comprises a first diluent in about 10% to about 60% by weight of the dosage form. In some embodiments, the pharmaceutical composition comprises a second diluent in about 10% to about 40% by weight of the dosage form. In some embodiments, the pharmaceutical composition comprises a binder in about 1% to about 10% by weight of the dosage form. In some embodiments, the pharmaceutical composition comprises a surfactant in about 0.5% to about 5% by weight of the dosage form. In some embodiments, the pharmaceutical composition comprises a disintegrant in about 1% to about 10% by weight of the dosage form. In some embodiments, the pharmaceutical composition comprises a glidant in about 0.5% to about 5% by weight of the dosage form. In some embodiments, the pharmaceutical composition comprises a lubricant in about 0.5% to about 5%.

In some embodiments, the pharmaceutical composition comprises a first diluent in about 35% to about 50% by weight of the dosage form. In some embodiments, the pharmaceutical composition comprises a second diluent in about 15% to about 35% by weight of the dosage form. In some embodiments, the pharmaceutical composition comprises a binder in about 1% to about 5% by weight of the dosage form. In some embodiments, the pharmaceutical composition comprises a surfactant in about 0.5% to about 2% by weight of the dosage form. In some embodiments, the pharmaceutical composition comprises a disintegrant in about 2% to about 6% by weight of the dosage form. In some embodiments, the pharmaceutical composition comprises a glidant in about 0.5% to about 3% by weight of the dosage form. In some embodiments, the pharmaceutical composition comprises a lubricant in about 0.5% to about 3% by weight of the dosage form.

### Amounts

In some embodiments, the pharmaceutical composition comprises between about 50 mg and about 200 mg. In some embodiments, the pharmaceutical composition comprises about 50 mg. In some embodiments, the pharmaceutical composition comprises about 200 mg. In some embodiments, the pharmaceutical composition comprises between about 1 mg and about 500 mg of the crystalline composition comprising Compound 1 (e.g., comprising Formula I). In some embodiments, the pharmaceutical composition comprises between about 1 mg and about 450 mg of the crystalline composition comprising Compound 1 (e.g., comprising Formula I). In some embodiments, the pharmaceutical composition comprises between about 1 mg and about 400 mg of the crystalline composition comprising Compound 1 (e.g., comprising Formula I). In some embodiments, the pharmaceutical composition comprises between about 1 mg and about 350 mg of the crystalline composition comprising Compound 1 (e.g., comprising Formula I). In some embodiments, the pharmaceutical composition comprises between about 1 mg and about 300 mg of the crystalline composition comprising Compound 1 (e.g., comprising Formula I). In some embodiments, the pharmaceutical composition comprises between about 1 mg and about 250 mg of the crystalline composition comprising Compound 1 (e.g., comprising Formula I). In some embodiments, the pharmaceutical composition comprises between about 1 mg and about 200 mg of the crystalline composition comprising Compound 1 (e.g., comprising Formula I). In some embodiments, the pharmaceutical composition comprises between about 1 mg and about 175 mg of the crystalline composition comprising Compound 1 (e.g., comprising Formula I). In some embodiments, the pharmaceutical composition comprises between about 1 mg and about 150 mg of the crystalline composition comprising Compound 1 (e.g., comprising Formula I). In some embodiments, the pharmaceutical composition comprises between about 1 mg and about 125 mg of the crystalline composition comprising Compound 1 (e.g., comprising Formula I). In some embodiments, the pharmaceutical composition comprises between about 1 mg and about 100 mg of the crystalline composition comprising Compound 1 (e.g., comprising Formula I). In some embodiments, the pharmaceutical composition comprises between about 1 mg and about 90 mg of the crystalline comprising Compound 1 (e.g., composition comprising Formula I). In some embodiments, the pharmaceutical composition comprises between about 1 mg and about 80 mg of the crystalline composition comprising Compound 1 (e.g., comprising Formula I). In some embodiments, the pharmaceutical composition comprises between about 1 mg and about 70 mg of the crystalline composition comprising Compound 1 (e.g., comprising Formula I). In some embodiments, the pharmaceutical composition comprises between about 1 mg and about 60 mg of the crystalline composition comprising Compound 1 (e.g., comprising Formula I). In some embodiments, the pharmaceutical composition comprises between about 1 mg and about 50 mg of the crystalline composition comprising Compound 1 (e.g., comprising Formula I). In some embodiments, the pharmaceutical composition comprises between about 1 mg and about 40 mg of the crystalline composition comprising Compound 1 (e.g., comprising Formula I). In some embodiments, the pharmaceutical composition comprises between about 1 mg and about 30 mg of the crystalline composition comprising Compound 1 (e.g., comprising Formula I). In some embodiments, the pharmaceutical composition comprises between about 1 mg and about 20 mg of the crystalline composition comprising Compound 1 (e.g., comprising Formula I). In some embodiments, the pharmaceutical composition comprises between about 1 mg and about 10 mg of the crystalline composition comprising Compound 1 (e.g., comprising Formula I). In some embodiments, the pharmaceutical composition comprises between about 1 mg and about 9 mg of the crystalline composition comprising Compound 1 (e.g., comprising Formula I). In some embodiments, the pharmaceutical composition comprises between about 1 mg and about 8 mg of the crystalline composition comprising Compound 1 (e.g., comprising Formula I). In some embodiments, the pharmaceutical composition comprises between about 1 mg and about 7 mg of the crystalline composition comprising Compound 1 (e.g., comprising Formula I). In some embodiments, the pharmaceutical composition comprises between about 1 mg and about 6 mg of the crystalline composition comprising Compound 1 (e.g., comprising Formula I). In some embodiments, the pharmaceutical composition comprises between about 1 mg and about 5 mg of the crystalline composition comprising Compound 1 (e.g., comprising Formula I). In some embodiments, the pharmaceutical composition comprises between about 1 mg and about 4 mg of the crystalline composition comprising Compound 1 (e.g., comprising Formula I). In some embodiments, the pharmaceutical composition comprises between about 1 mg and about 3 mg of the crystalline composition comprising Compound 1 (e.g., comprising Formula I). In some embodiments, the pharmaceutical composition comprises between about 1 mg and about 2 mg of the crystalline composition comprising Compound 1 (e.g., comprising Formula I).

In some embodiments, the pharmaceutical composition comprises between about 500 mg of the crystalline composition comprising Compound 1 (e.g., comprising Formula I). In some embodiments, the pharmaceutical composition comprises between about 450 mg of the crystalline composition comprising Compound 1 (e.g., comprising Formula I). In some embodiments, the pharmaceutical composition comprises between about 400 mg of the crystalline composition comprising Compound 1 (e.g., comprising Formula I). In some embodiments, the pharmaceutical composition comprises between about 350 mg of the crystalline composition comprising Compound 1 (e.g., comprising Formula I). In some embodiments, the pharmaceutical composition comprises between about 300 mg of the crystalline composition comprising Compound 1 (e.g., comprising Formula I. In some embodiments, the pharmaceutical composition comprises between about 250 mg of the crystalline composition comprising Compound 1 (e.g., comprising Formula I). In some embodiments, the pharmaceutical composition comprises between about 200 mg of the crystalline composition comprising Compound 1 (e.g., comprising Formula I). In some embodiments, the pharmaceutical composition comprises between about 150 mg of the crystalline composition comprising Compound 1 (e.g., comprising Formula I). In some embodiments, the pharmaceutical composition comprises between about 100 mg of the crystalline comprising Compound 1 (e.g., composition comprising Formula I). In some embodiments, the pharmaceutical composition comprises between about 90 mg of the crystalline composition comprising Compound 1 (e.g., comprising Formula I). In some embodiments, the pharmaceutical composition comprises between about 80 mg of the crystalline composition comprising Compound 1 (e.g., comprising Formula I). In some embodiments, the pharmaceutical composition comprises between about 70 mg of the crystalline composition comprising Compound 1 (e.g., comprising Formula I). In some embodiments, the pharmaceutical composition comprises between about 60 mg of the crystalline composition comprising Compound 1 (e.g., comprising Formula I). In some embodiments, the pharmaceutical composition comprises between about 50 mg of the crystalline composition comprising Compound 1 (e.g., comprising Formula I). In some embodiments, the pharmaceutical composition comprises between about 40 mg of the crystalline composition comprising Compound 1 (e.g., comprising Formula I). In some embodiments, the pharmaceutical composition comprises between about 30 mg of the crystalline composition comprising Compound 1 (e.g., comprising Formula I). In some embodiments, the pharmaceutical composition comprises between about 20 mg of the crystalline composition comprising Compound 1 (e.g., comprising Formula I). In some embodiments, the pharmaceutical composition comprises between about 10 mg of the crystalline composition comprising Compound 1 (e.g., comprising Formula I). In some embodiments, the pharmaceutical composition comprises between about 5 mg of the crystalline composition comprising Compound 1 (e.g., comprising Formula I). In some embodiments, the pharmaceutical composition comprises between about 1 mg of the crystalline composition comprising Compound 1 (e.g., comprising Formula I).

In some embodiments, the pharmaceutical composition (e.g., crystalline composition) comprising Compound 1 comprises between about 50 mg and about 200 mg of Compound 1. In some embodiments, the pharmaceutical composition (e.g., crystalline composition) comprising Compound 1 comprises about 50 of Compound 1. In some embodiments, the pharmaceutical composition (e.g., crystalline composition) comprising Compound 1 comprises about 100 of Compound 1. In some embodiments, the pharmaceutical composition (e.g., crystalline composition) comprising Compound 1 comprises about 200 of Compound 1. In some embodiments, the pharmaceutical composition comprises between about 1 mg and about 500 mg of Compound 1. In some embodiments, the pharmaceutical composition comprises between about 1 mg and about 450 mg of Compound 1. In some embodiments, the pharmaceutical composition comprises between about 1 mg and about 400 mg Compound 1. In some embodiments, the pharmaceutical composition comprises between about 1 mg and about 350 mg of Compound 1. In some embodiments, the pharmaceutical composition comprises between about 1 mg and about 300 mg of Compound 1. In some embodiments, the pharmaceutical composition comprises between about 1 mg and about 250 mg of Compound 1. In some embodiments, the pharmaceutical composition comprises between about 1 mg and about 200 mg of Compound 1. In some embodiments, the pharmaceutical composition comprises between about 1 mg and about 175 mg of Compound 1. In some embodiments, the pharmaceutical composition comprises between about 1 mg and about 150 mg of Compound 1. In some embodiments, the pharmaceutical composition comprises between about 1 mg and about 125 mg of Compound 1. In some embodiments, the pharmaceutical composition comprises between about 1 mg and about 100 mg of Compound 1. In some embodiments, the pharmaceutical composition comprises between about 1 mg and about 90 mg of Compound 1. In some embodiments, the pharmaceutical composition comprises between about 1 mg and about 80 mg of Compound 1. In some embodiments, the pharmaceutical composition comprises between about 1 mg and about 70 mg of Compound 1. In some embodiments, the pharmaceutical composition comprises between about 1 mg and about 60 mg of Compound 1. In some embodiments, the pharmaceutical composition comprises between about 1 mg and about 50 mg of Compound 1. In some embodiments, the pharmaceutical composition comprises between about 1 mg and about 40 mg of Compound 1. In some embodiments, the pharmaceutical composition comprises between about 1 mg and about 30 mg of Compound 1. In some embodiments, the pharmaceutical composition comprises between about 1 mg and about 20 mg of Compound 1. In some embodiments, the pharmaceutical composition comprises between about 1 mg and about 10 mg of Compound 1. In some embodiments, the pharmaceutical composition comprises between about 1 mg and about 9 mg of Compound 1. In some embodiments, the pharmaceutical composition comprises between about 1 mg and about 8 mg of Compound 1. In some embodiments, the pharmaceutical composition comprises between about 1 mg and about 7 mg of Compound 1. In some embodiments, the pharmaceutical composition comprises between about 1 mg and about 6 mg of Compound 1. In some embodiments, the pharmaceutical composition comprises between about 1 mg and about 5 mg of Compound 1. In some embodiments, the pharmaceutical composition comprises between about 1 mg and about 4 mg of Compound 1. In some embodiments, the pharmaceutical composition comprises between about 1 mg and about 3 mg of Compound 1. In some embodiments, the pharmaceutical composition comprises between about 1 mg and about 2 mg of Compound 1.

In some embodiments, the pharmaceutical composition comprises about 500 mg of Compound 1. In some embodiments, the pharmaceutical composition comprises about 450 mg of Compound 1. In some embodiments, the pharmaceutical composition comprises about 400 mg of Compound 1. In some embodiments, the pharmaceutical composition comprises about 350 mg of Compound 1. In some embodiments, the pharmaceutical composition comprises about 300 mg of Compound 1. In some embodiments, the pharmaceutical composition comprises about 250 mg of Compound 1. In some embodiments, the pharmaceutical composition comprises about 200 mg of Compound 1. In some embodiments, the pharmaceutical composition comprises about 150 mg of Compound 1. In some embodiments, the pharmaceutical composition comprises about 100 mg of Compound 1. In some embodiments, the pharmaceutical composition comprises about 90 mg of Compound 1. In some embodiments, the pharmaceutical composition comprises about 80 mg of Compound 1. In some embodiments, the pharmaceutical composition comprises about 70 mg of Compound 1. In some embodiments, the pharmaceutical composition comprises about 60 mg of Compound 1. In some embodiments, the pharmaceutical composition comprises about 50 mg of Compound 1. In some embodiments, the pharmaceutical composition comprises about 40 mg of Compound 1. In some embodiments, the pharmaceutical composition comprises about 30 mg of Compound 1. In some embodiments, the pharmaceutical composition comprises about 20 mg of Compound 1. In some embodiments, the pharmaceutical composition comprises about 10 mg of Compound 1. In some embodiments, the pharmaceutical composition comprises about 5 mg of Compound 1. In some embodiments, the pharmaceutical composition comprises about 1 mg Compound 1.

In some embodiments, the pharmaceutical composition comprises Compound I in about 25% w/w. In some embodiments, the pharmaceutical composition comprises Compound I in about 10% to about 50% w/w. In some embodiments, the pharmaceutical composition comprises Compound I in about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50% w/w. In some embodiments, the pharmaceutical composition comprises Compound I in about 10%, about 15%, about 200%, about 25%, about 30%, about 35%, about 40%, about 45%, or about 50% w/w. In some embodiments, the pharmaceutical composition comprises Compound I in at least about 10%, about 15%, about 20%, about 25%, about 30%, about 35%, about 40%, or about 45% w/w. In some embodiments, the pharmaceutical composition comprises Compound I in at most about 15%, about 20%, about 25%, about 30%, about 35%, about 40%, about 45%, or about 50% w/w.

In some embodiments, the pharmaceutical composition comprises a diluent in an amount that is between about 10 mg to about 500 mg. In some embodiments, the pharmaceutical composition comprises a diluent in an amount that is between about 10 mg to about 450 mg. In some embodiments, the pharmaceutical composition comprises a diluent in an amount that is between about 10 mg to about 400 mg. In some embodiments, the pharmaceutical composition comprises a diluent in an amount that is between about 10 mg to about 350 mg. In some embodiments, the pharmaceutical composition comprises a diluent in an amount that is between about 10 mg to about 300 mg. In some embodiments, the pharmaceutical composition comprises a diluent in an amount that is between about 10 mg to about 250 mg. In some embodiments, the pharmaceutical composition comprises a diluent in an amount that is between about 10 mg to about 200 mg. In some embodiments, the pharmaceutical composition comprises a diluent in an amount that is between about 10 mg to about 150 mg. In some embodiments, the pharmaceutical composition comprises a diluent in an amount that is between about 10 mg to about 100 mg. In some embodiments, the pharmaceutical composition comprises a diluent in an amount that is between about 10 mg to about 90 mg. In some embodiments, the pharmaceutical composition comprises a diluent in an amount that is between about 10 mg to about 80 mg. In some embodiments, the pharmaceutical composition comprises a diluent in an amount that is between about 10 mg to about 70 mg. In some embodiments, the pharmaceutical composition comprises a diluent in an amount that is between about 10 mg to about 60 mg. In some embodiments, the pharmaceutical composition comprises a diluent in an amount that is between about 10 mg to about 50 mg. In some embodiments, the pharmaceutical composition comprises a diluent in an amount that is between about 10 mg to about 40 mg. In some embodiments, the pharmaceutical composition comprises a diluent in an amount that is between about 10 mg to about 30 mg. In some embodiments, the pharmaceutical composition comprises a diluent in an amount that is between about 10 mg to about 20 mg.

In some embodiments, the pharmaceutical composition comprises a diluent in an amount that is about 300 mg. In some embodiments, the pharmaceutical composition comprises a diluent in an amount that is about 200 mg. In some embodiments, the pharmaceutical composition comprises a diluent in an amount that is about 100 mg. In some embodiments, the pharmaceutical composition comprises a diluent in an amount that is about 90 mg. In some embodiments, the pharmaceutical composition comprises a diluent in an amount that is about 80 mg. In some embodiments, the pharmaceutical composition comprises a diluent in an amount that is about 70 mg. In some embodiments, the pharmaceutical composition comprises a diluent in an amount that is about 60 mg. In some embodiments, the pharmaceutical composition comprises a diluent in an amount that is about 50 mg. In some embodiments, the pharmaceutical composition comprises a diluent in an amount that is about 40 mg. In some embodiments, the pharmaceutical composition comprises a diluent in an amount that is about 30 mg. In some embodiments, the pharmaceutical composition comprises a diluent in an amount that is about 20 mg. In some embodiments, the pharmaceutical composition comprises a diluent in an amount that is about 10 mg.

In some embodiments, the pharmaceutical composition comprises a first diluent in an amount that is about 300 mg. In some embodiments, the pharmaceutical composition comprises a first diluent in an amount that is about 200 mg. In some embodiments, the pharmaceutical composition comprises a first diluent in an amount that is about 100 mg. In some embodiments, the pharmaceutical composition comprises a first diluent in an amount that is about 90 mg. In some embodiments, the pharmaceutical composition comprises a first diluent in an amount that is about 80 mg. In some embodiments, the pharmaceutical composition comprises a first diluent in an amount that is about 70 mg. In some embodiments, the pharmaceutical composition comprises a first diluent in an amount that is about 60 mg. In some embodiments, the pharmaceutical composition comprises a first diluent in an amount that is about 50 mg. In some embodiments, the pharmaceutical composition comprises a first diluent in an amount that is about 40 mg. In some embodiments, the pharmaceutical composition comprises a first diluent in an amount that is about 30 mg. In some embodiments, the pharmaceutical composition comprises a first diluent in an amount that is about 20 mg. In some embodiments, the pharmaceutical composition comprises a first diluent in an amount that is about 10 mg.

In some embodiments, the pharmaceutical composition comprises a second diluent in an amount that is about 300 mg. In some embodiments, the pharmaceutical composition comprises a second diluent in an amount that is about 200 mg. In some embodiments, the pharmaceutical composition comprises a second diluent in an amount that is about 100 mg. In some embodiments, the pharmaceutical composition comprises a second diluent in an amount that is about 90 mg. In some embodiments, the pharmaceutical composition comprises a second diluent in an amount that is about 80 mg. In some embodiments, the pharmaceutical composition comprises a second diluent in an amount that is about 70 mg. In some embodiments, the pharmaceutical composition comprises a second diluent in an amount that is about 60 mg. In some embodiments, the pharmaceutical composition comprises a second diluent in an amount that is about 50 mg. In some embodiments, the pharmaceutical composition comprises a second diluent in an amount that is about 40 mg. In some embodiments, the pharmaceutical composition comprises a second diluent in an amount that is about 30 mg. In some embodiments, the pharmaceutical composition comprises a second diluent in an amount that is about 20 mg. In some embodiments, the pharmaceutical composition comprises a second diluent in an amount that is about 10 mg.

In some embodiments, the pharmaceutical composition comprises a binder in an amount that is between about 1 mg and about 100 mg. In some embodiments, the pharmaceutical composition comprises a binder in an amount that is between about 1 mg and about 90 mg. In some embodiments, the pharmaceutical composition comprises a binder in an amount that is between about 1 mg and about 80 mg. In some embodiments, the pharmaceutical composition comprises a binder in an amount that is between about 1 mg and about 70 mg. In some embodiments, the pharmaceutical composition comprises a binder in an amount that is between about 1 mg and about 60 mg. In some embodiments, the pharmaceutical composition comprises a binder in an amount that is between about 1 mg and about 50 mg. In some embodiments, the pharmaceutical composition comprises a binder in an amount that is between about 1 mg and about 40 mg. In some embodiments, the pharmaceutical composition comprises a binder in an amount that is between about 1 mg and about 30 mg. In some embodiments, the pharmaceutical composition comprises a binder in an amount that is between about 1 mg and about 20 mg. In some embodiments, the pharmaceutical composition comprises a binder in an amount that is between about 1 mg and about 10 mg. In some embodiments, the pharmaceutical composition comprises a binder in an amount that is between about 1 mg and about 5 mg.

In some embodiments, the pharmaceutical composition comprises a binder in an amount that is about 100 mg. In some embodiments, the pharmaceutical composition comprises a binder in an amount that is about 90 mg. In some embodiments, the pharmaceutical composition comprises a binder in an amount that is about 80 mg. In some embodiments, the pharmaceutical composition comprises a binder in an amount that is about 70 mg. In some embodiments, the pharmaceutical composition comprises a binder in an amount that is about 60 mg. In some embodiments, the pharmaceutical composition comprises a binder in an amount that is about 50 mg. In some embodiments, the pharmaceutical composition comprises a binder in an amount that is about 40 mg. In some embodiments, the pharmaceutical composition comprises a binder in an amount that is about 30 mg. In some embodiments, the pharmaceutical composition comprises a binder in an amount that is about 20 mg. In some embodiments, the pharmaceutical composition comprises a binder in an amount that is about 10 mg. In some embodiments, the pharmaceutical composition comprises a binder in an amount that is about 9 mg. In some embodiments, the pharmaceutical composition comprises a binder in an amount that is about 8 mg. In some embodiments, the pharmaceutical composition comprises a binder in an amount that is about 7 mg. In some embodiments, the pharmaceutical composition comprises a binder in an amount that is about 6 mg. In some embodiments, the pharmaceutical composition comprises a binder in an amount that is about 5 mg. In some embodiments, the pharmaceutical composition comprises a binder in an amount that is about 4 mg. In some embodiments, the pharmaceutical composition comprises a binder in an amount that is about 3 mg. In some embodiments, the pharmaceutical composition comprises a binder in an amount that is about 2 mg. In some embodiments, the pharmaceutical composition comprises a binder in an amount that is about 1 mg.

In some embodiments, the pharmaceutical composition comprises a surfactant in an amount that is between about 1 mg and about 50 mg. In some embodiments, the pharmaceutical composition comprises a surfactant in an amount that is between about 1 mg and about 40 mg. In some embodiments, the pharmaceutical composition comprises a surfactant in an amount that is between about 1 mg and about 30 mg. In some embodiments, the pharmaceutical composition comprises a surfactant in an amount that is between about 1 mg and about 20 mg. In some embodiments, the pharmaceutical composition comprises a surfactant in an amount that is between about 1 mg and about 10 mg. In some embodiments, the pharmaceutical composition comprises a surfactant in an amount that is between about 1 mg and about 9 mg. In some embodiments, the pharmaceutical composition comprises a surfactant in an amount that is between about 1 mg and about 8 mg. In some embodiments, the pharmaceutical composition comprises a surfactant in an amount that is between about 1 mg and about 7 mg. In some embodiments, the pharmaceutical composition comprises a surfactant in an amount that is between about 1 mg and about 6 mg. In some embodiments, the pharmaceutical composition comprises a surfactant in an amount that is between about 1 mg and about 5 mg. In some embodiments, the pharmaceutical composition comprises a surfactant in an amount that is between about 1 mg and about 4 mg. In some embodiments, the pharmaceutical composition comprises a surfactant in an amount that is between about 1 mg and about 3 mg. In some embodiments, the pharmaceutical composition comprises a surfactant in an amount that is between about 1 mg and about 2 mg. In some embodiments, the pharmaceutical composition comprises a surfactant in an amount that is between about 1 mg and about 1.5 mg.

In some embodiments, the pharmaceutical composition comprises a surfactant in an amount that is about 50 mg. In some embodiments, the pharmaceutical composition comprises a surfactant in an amount that is about 40 mg. In some embodiments, the pharmaceutical composition comprises a surfactant in an amount that is about 30 mg. In some embodiments, the pharmaceutical composition comprises a surfactant in an amount that is about 20 mg. In some embodiments, the pharmaceutical composition comprises a surfactant in an amount that is about 10 mg. In some embodiments, the pharmaceutical composition comprises a surfactant in an amount that is about 9 mg. In some embodiments, the pharmaceutical composition comprises a surfactant in an amount that is about 8 mg. In some embodiments, the pharmaceutical composition comprises a surfactant in an amount that is about 7 mg. In some embodiments, the pharmaceutical composition comprises a surfactant in an amount that is about 6 mg. In some embodiments, the pharmaceutical composition comprises a surfactant in an amount that is about 5 mg. In some embodiments, the pharmaceutical composition comprises a surfactant in an amount that is about 4 mg. In some embodiments, the pharmaceutical composition comprises a surfactant in an amount that is about 3 mg. In some embodiments, the pharmaceutical composition comprises a surfactant in an amount that is about 2 mg. In some embodiments, the pharmaceutical composition comprises a surfactant in an amount that is about 1.75 mg. In some embodiments, the pharmaceutical composition comprises a surfactant in an amount that is about 1.5 mg. In some embodiments, the pharmaceutical composition comprises a surfactant in an amount that is about 1.25 mg. In some embodiments, the pharmaceutical composition comprises a surfactant in an amount that is about 1 mg.

In some embodiments, the pharmaceutical composition comprises a disintegrant in an amount that is between about 1 mg to about 100 mg. In some embodiments, the pharmaceutical composition comprises a disintegrant in an amount that is between about 1 mg to about 90 mg. In some embodiments, the pharmaceutical composition comprises a disintegrant in an amount that is between about 1 mg to about 80 mg. In some embodiments, the pharmaceutical composition comprises a disintegrant in an amount that is between about 1 mg to about 70 mg. In some embodiments, the pharmaceutical composition comprises a disintegrant in an amount that is between about 1 mg to about 60 mg. In some embodiments, the pharmaceutical composition comprises a disintegrant in an amount that is between about 1 mg to about 50 mg. In some embodiments, the pharmaceutical composition comprises a disintegrant in an amount that is between about 1 mg to about 40 mg. In some embodiments, the pharmaceutical composition comprises a disintegrant in an amount that is between about 1 mg to about 30 mg. In some embodiments, the pharmaceutical composition comprises a disintegrant in an amount that is between about 1 mg to about 20 mg. In some embodiments, the pharmaceutical composition comprises a disintegrant in an amount that is between about 1 mg to about 10 mg. In some embodiments, the pharmaceutical composition comprises a disintegrant in an amount that is between about 1 mg to about 9 mg. In some embodiments, the pharmaceutical composition comprises a disintegrant in an amount that is between about 1 mg to about 8 mg. In some embodiments, the pharmaceutical composition comprises a disintegrant in an amount that is between about 1 mg to about 7 mg. In some embodiments, the pharmaceutical composition comprises a disintegrant in an amount that is between about 1 mg to about 6 mg. In some embodiments, the pharmaceutical composition comprises a disintegrant in an amount that is between about 1 mg to about 5 mg. In some embodiments, the pharmaceutical composition comprises a disintegrant in an amount that is between about 1 mg to about 4 mg. In some embodiments, the pharmaceutical composition comprises a disintegrant in an amount that is between about 1 mg to about 3 mg. In some embodiments, the pharmaceutical composition comprises a disintegrant in an amount that is between about 1 mg to about 2 mg.

In some embodiments, the pharmaceutical composition comprises a disintegrant in an amount that is about 100 mg. In some embodiments, the pharmaceutical composition comprises a disintegrant in an amount that is about 90 mg. In some embodiments, the pharmaceutical composition comprises a disintegrant in an amount that is about 80 mg. In some embodiments, the pharmaceutical composition comprises a disintegrant in an amount that is about 70 mg. In some embodiments, the pharmaceutical composition comprises a disintegrant in an amount that is about 60 mg. In some embodiments, the pharmaceutical composition comprises a disintegrant in an amount that is about 50 mg. In some embodiments, the pharmaceutical composition comprises a disintegrant in an amount that is about 40 mg. In some embodiments, the pharmaceutical composition comprises a disintegrant in an amount that is about 30 mg. In some embodiments, the pharmaceutical composition comprises a disintegrant in an amount that is about 20 mg. In some embodiments, the pharmaceutical composition comprises a disintegrant in an amount that is about 10 mg. In some embodiments, the pharmaceutical composition comprises a disintegrant in an amount that is about 9 mg. In some embodiments, the pharmaceutical composition comprises a disintegrant in an amount that is about 8 mg. In some embodiments, the pharmaceutical composition comprises a disintegrant in an amount that is about 7 mg. In some embodiments, the pharmaceutical composition comprises a disintegrant in an amount that is about 6 mg. In some embodiments, the pharmaceutical composition comprises a disintegrant in an amount that is about 5 mg. In some embodiments, the pharmaceutical composition comprises a disintegrant in an amount that is about 4 mg. In some embodiments, the pharmaceutical composition comprises a disintegrant in an amount that is about 3 mg. In some embodiments, the pharmaceutical composition comprises a disintegrant in an amount that is about 2 mg. In some embodiments, the pharmaceutical composition comprises a disintegrant in an amount that is about 1 mg.

In some embodiments, the pharmaceutical composition comprises a glidant in an amount that is between about 1 mg and about 50 mg. In some embodiments, the pharmaceutical composition comprises a glidant in an amount that is between about 1 mg and about 45 mg. In some embodiments, the pharmaceutical composition comprises a glidant in an amount that is between about 1 mg and about 40 mg. In some embodiments, the pharmaceutical composition comprises a glidant in an amount that is between about 1 mg and about 35 mg. In some embodiments, the pharmaceutical composition comprises a glidant in an amount that is between about 1 mg and about 30 mg. In some embodiments, the pharmaceutical composition comprises a glidant in an amount that is between about 1 mg and about 25 mg. In some embodiments, the pharmaceutical composition comprises a glidant in an amount that is between about 1 mg and about 20 mg. In some embodiments, the pharmaceutical composition comprises a glidant in an amount that is between about 1 mg and about 15 mg. In some embodiments, the pharmaceutical composition comprises a glidant in an amount that is between about 1 mg and about 10 mg. In some embodiments, the pharmaceutical composition comprises a glidant in an amount that is between about 1 mg and about 5 mg. In some embodiments, the pharmaceutical composition comprises a glidant in an amount that is between about 1 mg and about 2 mg.

In some embodiments, the pharmaceutical composition comprises a glidant in an amount that is about 50 mg. In some embodiments, the pharmaceutical composition comprises a glidant in an amount that is about 40 mg. In some embodiments, the pharmaceutical composition comprises a glidant in an amount that is about 30 mg. In some embodiments, the pharmaceutical composition comprises a glidant in an amount that is about 20 mg. In some embodiments, the pharmaceutical composition comprises a glidant in an amount that is about 10 mg. In some embodiments, the pharmaceutical composition comprises a glidant in an amount that is about 9 mg. In some embodiments, the pharmaceutical composition comprises a glidant in an amount that is about 8 mg. In some embodiments, the pharmaceutical composition comprises a glidant in an amount that is about 7 mg. In some embodiments, the pharmaceutical composition comprises a glidant in an amount that is about 6 mg. In some embodiments, the pharmaceutical composition comprises a glidant in an amount that is about 5 mg. In some embodiments, the pharmaceutical composition comprises a glidant in an amount that is about 4 mg. In some embodiments, the pharmaceutical composition comprises a glidant in an amount that is about 3 mg. In some embodiments, the pharmaceutical composition comprises a glidant in an amount that is about 2 mg. In some embodiments, the pharmaceutical composition comprises a glidant in an amount that is about 1 mg.

In some embodiments, the pharmaceutical composition comprises a lubricant in an amount that is between about 1 mg and about 50 mg. In some embodiments, the pharmaceutical composition comprises a lubricant in an amount that is between about 1 mg and about 45 mg. In some embodiments, the pharmaceutical composition comprises a lubricant in an amount that is between about 1 mg and about 40 mg. In some embodiments, the pharmaceutical composition comprises a lubricant in an amount that is between about 1 mg and about 35 mg. In some embodiments, the pharmaceutical composition comprises a lubricant in an amount that is between about 1 mg and about 30 mg. In some embodiments, the pharmaceutical composition comprises a lubricant in an amount that is between about 1 mg and about 25 mg. In some embodiments, the pharmaceutical composition comprises a lubricant in an amount that is between about 1 mg and about 20 mg. In some embodiments, the pharmaceutical composition comprises a lubricant in an amount that is between about 1 mg and about 15 mg. In some embodiments, the pharmaceutical composition comprises a lubricant in an amount that is between about 1 mg and about 10 mg. In some embodiments, the pharmaceutical composition comprises a lubricant in an amount that is between about 1 mg and about 5 mg. In some embodiments, the pharmaceutical composition comprises a lubricant in an amount that is between about 1 mg and about 2 mg.

In some embodiments, the pharmaceutical composition comprises a lubricant in an amount that is about 50 mg. In some embodiments, the pharmaceutical composition comprises a lubricant in an amount that is about 40 mg. In some embodiments, the pharmaceutical composition comprises a lubricant in an amount that is about 30 mg. In some embodiments, the pharmaceutical composition comprises a lubricant in an amount that is about 20 mg. In some embodiments, the pharmaceutical composition comprises a lubricant in an amount that is about 10 mg. In some embodiments, the pharmaceutical composition comprises a lubricant in an amount that is about 9 mg. In some embodiments, the pharmaceutical composition comprises a lubricant in an amount that is about 8 mg. In some embodiments, the pharmaceutical composition comprises a lubricant in an amount that is about 7 mg. In some embodiments, the pharmaceutical composition comprises a lubricant in an amount that is about 6 mg. In some embodiments, the pharmaceutical composition comprises a lubricant in an amount that is about 5 mg. In some embodiments, the pharmaceutical composition comprises a lubricant in an amount that is about 4 mg. In some embodiments, the pharmaceutical composition comprises a lubricant in an amount that is about 3 mg. In some embodiments, the pharmaceutical composition comprises a lubricant in an amount that is about 2 mg. In some embodiments, the pharmaceutical composition comprises a lubricant in an amount that is about 1 mg.

In some embodiments, the pharmaceutical composition comprises a first diluent in an amount that is between about 1 mg to about 200 mg by weight of the dosage form. In some embodiments, the pharmaceutical composition comprises a second diluent in an amount that is between about 1 mg to about 200 mg by weight of the dosage form. In some embodiments, the pharmaceutical composition comprises a binder in an amount that is between about 1 mg to about 100 mg by weight of the dosage form. In some embodiments, the pharmaceutical composition comprises a surfactant in an amount that is between about 1 mg to about 50 mg by weight of the dosage form. In some embodiments, the pharmaceutical composition comprises a disintegrant in an amount that is between about 1 mg to about 100 mg by weight of the dosage form. In some embodiments, the pharmaceutical composition comprises a glidant in an amount that is between about 1 mg to about 50 mg by weight of the dosage form. In some embodiments, the pharmaceutical composition comprises a lubricant in an amount that is between about 1 mg to about 50 mg by weight of the dosage form.

In some embodiments, the pharmaceutical composition comprises a first diluent in an amount that is between about 1 mg to about 100 mg by weight of the dosage form. In some embodiments, the pharmaceutical composition comprises a second diluent in an amount that is between about 1 mg to about 100 mg by weight of the dosage form. In some embodiments, the pharmaceutical composition comprises a binder in an amount that is between about 1 mg and 20 mg. In some embodiments, the pharmaceutical composition comprises a surfactant in an amount that is between about 1 mg to about 10 mg by weight of the dosage form. In some embodiments, the pharmaceutical composition comprises a disintegrant in an amount that is between about 1 mg to about 20 mg by weight of the dosage form. In some embodiments, the pharmaceutical composition comprises a glidant in an amount that is between about 1 mg to about 20 mg by weight of the dosage form. In some embodiments, the pharmaceutical composition comprises a lubricant in an amount that is between about 1 mg to about 20 mg by weight of the dosage form.

### Particle Size

In some embodiments, the pharmaceutical composition (e.g., comprising the crystalline composition comprising Compound 1 (e.g., comprising Formula I)) is in the form of microparticles. In some embodiments, microparticles of the crystalline composition comprising Compound 1 (e.g., comprising Formula I) have an average size from about 1 μm to about 100 μm. In some embodiments, microparticles of the crystalline composition have an average size from about 1 μm to about 90 μm. In some embodiments, microparticles of the crystalline composition have an average size from about 1 μm to about 80 μm. In some embodiments, microparticles of the crystalline composition have an average size from about 1 μm to about 70 μm. In some embodiments, microparticles of the crystalline composition have an average size from about 1 μm to about 60 μm. In some embodiments, microparticles of the crystalline composition have an average size from about 1 μm to about 50 μm. In some embodiments, microparticles of the crystalline composition have an average size from about 1 μm to about 40 μm. In some embodiments, microparticles of the crystalline composition have an average size from about 1 μm to about 30 μm. In some embodiments, microparticles of the crystalline composition have an average size from about 1 μm to about 20 μm. In some embodiments, microparticles of the crystalline composition have an average size from about 1 μm to about 10 μm. In some embodiments, microparticles of the crystalline composition have an average size from about 1 μm to about 5 μm. In some embodiments, microparticles of the crystalline composition have an average size from about 100 μm. In some embodiments, microparticles of the crystalline composition have an average size from about 90 μm. In some embodiments, microparticles of the crystalline composition have an average size from about 80 μm. In some embodiments, microparticles of the crystalline composition have an average size from about 70 μm. In some embodiments, microparticles of the crystalline composition have an average size from about 60 μm. In some embodiments, microparticles of the crystalline composition have an average size from about 50 μm. In some embodiments, microparticles of the crystalline composition have an average size from about 40 μm. In some embodiments, microparticles of the crystalline composition have an average size from about 30 μm. In some embodiments, microparticles of the crystalline composition have an average size from about 20 μm. In some embodiments, microparticles of the crystalline composition have an average size from about 10 μm. In some embodiments, microparticles of the crystalline composition have an average size from about 9 μm. In some embodiments, microparticles of the crystalline composition have an average size from about 8 μm. In some embodiments, microparticles of the crystalline composition have an average size from about 7 μm. In some embodiments, microparticles of the crystalline composition have an average size from about 6 μm. In some embodiments, microparticles of the crystalline composition have an average size from about 5 μm. In some embodiments, microparticles of the crystalline composition have an average size from about 4 μm. In some embodiments, microparticles of the crystalline composition have an average size from about 3 μm. In some embodiments, microparticles of the crystalline composition have an average size from about 2 μm. In some embodiments, microparticles of the crystalline composition have an average size from about 1 μm. In some embodiments, microparticles of the crystalline composition have an average size less than about 100 μm. In some embodiments, microparticles of the crystalline composition have an average size less than about 90 μm. In some embodiments, microparticles of the crystalline composition have an average size less than about 80 μm. In some embodiments, microparticles of the crystalline composition have an average size less than about 70 μm. In some embodiments, microparticles of the crystalline composition have an average size less than about 60 μm. In some embodiments, microparticles of the crystalline composition have an average size less than about 50 μm. In some embodiments, microparticles of the crystalline composition have an average size less than about 40 μm. In some embodiments, microparticles of the crystalline composition have an average size less than about 30 μm. In some embodiments, microparticles of the crystalline composition have an average size less than about 20 μm. In some embodiments, microparticles of the crystalline composition have an average size less than about 10 μm. In some embodiments, microparticles of the crystalline composition have an average size less than about 5 μm.

In some embodiments, the pharmaceutical composition comprises the crystalline composition comprising Compound 1 (e.g., comprising Formula I), wherein the crystalline composition has a D90 from about 1 μm to about 50 μm. In some embodiments, the crystalline composition has a D90 from about 1 μm to about 20 μm, from about 2 μm to about 19 μm, from about 3 μm to about 18 μm, from about 4 μm to about 17 μm, from about 5 μm to about 16 μm, from about 6 μm to about 15 μm, from about 7 μm to about 14 μm, from about 8 μm to about 13 μm, from about 9 μm to about 13 μm, or from about 8 μm to about 12 μm. In some embodiments, the pharmaceutical composition comprises the crystalline composition comprising Compound 1 (e.g., comprising Formula I), wherein the crystalline composition has a D90 of more than about 1 μm, more than about 2 μm, more than about 3 μm, more than about 4 μ, more than about 5 μm, more than about 6 μm, more than about 7 μm, more than about 8 μm, more than about 9 μm, more than about 10 μm, more than about 11 μm, more than about 12 μm, more than about 13 μm, more than about 14 μm more than about 15 μm more than about 16 μm, more than about 17 μm, more than about 18 μm, or more than about 19 μm. In some embodiments, the crystalline composition has a D90 of less than about 20 μm, less than about 19 μm, less than about 18 μm, less than about 17 μm, less than about 16 μm, less than about 15 μm, less than about 14 μm, less than about 13 μm, less than about 12 μm, or less than about 11 μm.

In some embodiments, the pharmaceutical composition comprising Compound 1 is in the form of microparticles. In some embodiments, microparticles of Compound 1 have an average size from about 1 μm to about 100 μm. In some embodiments, microparticles of Compound 1 have an average size from about 1 μm to about 90 μm. In some embodiments, microparticles of Compound 1 have an average size from about 1 μm to about 80 μm. In some embodiments, microparticles of Compound 1 have an average size from about 1 μm to about 70 μm. In some embodiments, microparticles of Compound 1 have an average size from about 1 μm to about 60 μm. In some embodiments, microparticles of Compound 1 have an average size from about 1 μm to about 50 μm. In some embodiments, microparticles of Compound 1 have an average size from about 1 μm to about 40 μm. In some embodiments, microparticles of Compound 1 have an average size from about 1 μm to about 30 μm. In some embodiments, microparticles of Compound 1 have an average size from about 1 μm to about 20 μm. In some embodiments, microparticles of Compound 1 have an average size from about 1 μm to about 10 μm. In some embodiments, microparticles of Compound 1 have an average size from about 1 μm to about 5 μm. In some embodiments, microparticles of Compound 1 have an average size from about 100 μm. In some embodiments, microparticles of Compound 1 have an average size from about 90 μm. In some embodiments, microparticles of Compound 1 have an average size from about 80 μm. In some embodiments, microparticles of Compound 1 have an average size from about 70 μm. In some embodiments, microparticles of Compound 1 have an average size from about 60 μm. In some embodiments, microparticles of Compound 1 have an average size from about 50 μm. In some embodiments, microparticles of Compound 1 have an average size from about 40 μm. In some embodiments, microparticles of Compound 1 have an average size from about 30 μm. In some embodiments, microparticles of Compound 1 have an average size from about 20 μm. In some embodiments, microparticles of Compound 1 have an average size from about 10 μm. In some embodiments, microparticles of Compound 1 have an average size from about 9 μm. In some embodiments, microparticles of Compound 1 have an average size from about 8 μm. In some embodiments, microparticles of Compound 1 have an average size from about 7 μm. In some embodiments, microparticles of Compound 1 have an average size from about 6 μm. In some embodiments, microparticles of Compound 1 have an average size from about 5 μm. In some embodiments, microparticles of Compound 1 have an average size from about 4 μm. In some embodiments, microparticles of Compound 1 have an average size from about 3 μm. In some embodiments, microparticles of Compound 1 have an average size from about 2 μm. In some embodiments, microparticles of Compound 1 have an average size from about 1 μm. In some embodiments, microparticles of Compound 1 have an average size less than about 100 μm. In some embodiments, microparticles of Compound 1 have an average size less than about 90 μm. In some embodiments, microparticles of Compound 1 have an average size less than about 80 μm. In some embodiments, microparticles of Compound 1 have an average size less than about 70 μm. In some embodiments, microparticles of Compound 1 have an average size less than about 60 μm. In some embodiments, microparticles of Compound 1 have an average size less than about 50 μm. In some embodiments, microparticles of Compound 1 have an average size less than about 40 μm. In some embodiments, microparticles of Compound 1 have an average size less than about 30 μm. In some embodiments, microparticles of Compound 1 have an average size less than about 20 μm. In some embodiments, microparticles of Compound 1 have an average size less than about 10 μm. In some embodiments, microparticles of Compound 1 have an average size less than about 5 μm.

In some embodiments, the pharmaceutical composition comprises Compound 1, wherein Compound 1 has a D90 from about 1 μm to about 50 μm. In some embodiments, Compound 1 has a D90 from about 1 μm to about 20 μm, from about 2 μm to about 19 μm, from about 3 μm to about 18 μm, from about 4 μm to about 17 μm, from about 5 μm to about 16 μm, from about 6 μm to about 15 μm, from about 7 μm to about 14 μm, from about 8 μm to about 13 μm, from about 9 μm to about 13 μm, or from about 8 μm to about 12 μm. In some embodiments, the pharmaceutical composition comprises Compound 1, wherein Compound 1 has a D90 of more than about 1 μm, more than about 2 μm, more than about 3 μm, more than about 4 μ, more than about 5 μm, more than about 6 μm, more than about 7 μm, more than about 8 μm, more than about 9 μm, more than about 10 μm, more than about 11 μm, more than about 12 μm, more than about 13 μm, more than about 14 μm more than about 15 μm more than about 16 μm, more than about 17 μm, more than about 18 μm, or more than about 19 μm. In some embodiments, Compound 1 has a D90 of less than about 20 μm, less than about 19 μm, less than about 18 μm, less than about 17 μm, less than about 16 μm, less than about 15 μm, less than about 14 μm, less than about 13 μm, less than about 12 μm, or less than about 11 μm.

### Methods of Preparing the Crystalline Composition Comprising Compound I (e.g., Comprising Formula I)

Isolation and purification of the chemical entities and intermediates described herein can be performed, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples below. However, other equivalent separation or isolation procedures can also be used. Prior to crystallization, Compound 1 may be isolated in about 50% chemical purity, 55% chemical purity, 60% chemical purity, 65% chemical purity, 70% chemical purity, 75% chemical purity, 80% chemical purity, 90% chemical purity, 91% chemical purity, 92% purity, 93% chemical purity, 94% chemical purity, 95% chemical purity, 96% chemical purity, 97% chemical purity, 98% chemical purity, 99% chemical purity, about 98% chemical purity, or about 100% chemical purity.

In some embodiments, the crystalline composition comprising Compound I (e.g., comprising Formula I) disclosed herein are obtained by crystallizing Compound 1 with a chemical purity of less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 78%, less than about 76%, less than about 74%, less than about 72%, or less than about 70%. In some embodiments, the crystalline composition comprising Compound I (e.g., comprising Formula I) is obtained by crystallizing Compound 1 with a chemical purity in the range of about 70% to about 99%, 80% to about 96%, about 85% to about 96%, about 90% to about 96%, about 80% to 98%, about 85% to about 98%, about 90% to about 98%, about 92% to about 98%, about 94% to 98%, or about 96% to about 98%.

### Preparation of Crystalline Form I

In some aspects, the present disclosure provides a method of preparing a crystalline composition comprising Compound I (e.g., comprising Formula I), wherein the method comprises:
(a) dissolving 4-(4-chloro-5-(2,5-dimethyl-7-(pentan-3-yl)pyrazolo[1,5-a]pyrimidin-3-yl)thiazol-2-yl)morpholine (Compound 1) and an acid (e.g., oxalic acid) in a solvent;
(b) heating the solution or slurry from step (a); and
(c) crystallizing the solution or slurry obtained in step (b) to obtain the crystalline composition comprising Compound I (e.g., comprising Formula I).

In some embodiments, the solvent in step (a) comprises acetone, heptane, water, 2-butanol, ethyl acetate, 2-propanol, methyl tert-butyl ether, ethanol, methyl ethyl ketone, 1-pentanol, or a combination thereof. In some embodiments, the solvent in step (a) is heptane. In some embodiments, the solvent in step (a) is methyl tert-butyl ether or methyl ethyl ketone. In some embodiments, the solvent in step (a) is a mixture of heptane and methyl ethyl ketone.

In some embodiments, step (b) is heated at about 50° C. In some embodiments, step (b) is heated to at least about 50° C.

In some embodiments, the concentration of the solution comprising Compound 1 obtained in step (a) is between about 20 mg/mL and about 300 mg/mL. In some embodiments, the concentration of the solution comprising Compound 1 obtained in step (a) is between about 40 mg/mL and about 250 mg/mL. In some embodiments, the concentration of the solution comprising Compound 1 obtained in step (a) is between about 100 mg/mL and about 200 mg/mL. In some embodiments, the concentration of the solution comprising Compound 1 obtained in step (a) is between 125 mg/mL and about 175 mg/mL.

In various embodiments, the method of preparing the crystalline composition comprising Compound I (e.g., comprising Formula I) involves recrystallization of Compound 1 from a binary, tertiary, or greater solvent system, collectively understood as a multi-solvent system. In some embodiments, the method of preparing the crystalline composition comprising Compound I (e.g., comprising Formula I) involves crystallization from a mono- or multi-solvent system, where the crystallization involves dissolving Compound 1 in the mono- or multi-solvent system at a temperature above ambient temperature. In some embodiments, the dissolving of Compound 1 in the mono- or multi-solvent system is performed at a temperature of about 0-90° C., 5-90° C., 10-90° C., 15-90° C., 20-90° C., 25-90° C., 30-90° C., 35-90° C., 40-90° C., 45-90° C., 50-90° C., 55-90° C., 60-90° C., 65-90° C., 70-90° C., 75-90° C., 80-90° C., 85-90° C., 0-80° C., 5-80° C., 10-80° C., 20-80° C., 30-80° C., 40-80° C., 50-80° C., 60-80° C., 70-80° C., 0-70° C., 5-70° C., 10-70° C., 15-70° C., 20-70° C., 30-70° C., 40-70° C., 50-70° C., 60-70° C., 0-60° C., 10-60° C., 20-60° C., 30-60° C., 40-60° C., or 50-60° C.

In various embodiments, the crystallization further involves actively heating the solution containing the dissolved Compound 1, for example to a temperature of about 40-100° C., 40-90° C., 40-80° C., 40-70° C., 40-60° C., 40-50° C., 50-100° C., 50-90° C., 50-80° C., 50-70° C., 50-60° C., 60-100° C., 60-90° C., 60-80° C., 60-70° C., 70-100° C., 70-90° C., 70-80° C., 80-100° C., or 80-90° C. In various embodiments, the solution containing the dissolved Compound 1 is maintained at the heated temperature for a period of time, for example for about 30 min, about 1 h, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h or more.

In various embodiments, the crystallization further involves actively cooling the heated solution containing the dissolved Compound 1, for example to a temperature of about 0-40° C., 0-30° C., 0-20° C., 0-10° C., 10-40° C., 10-30° C., 10-20° C., 20-40° C., 20-30° C., 20-10° C., or 30° C.-40° C. In some embodiments, the crystallization further involves actively cooling the heated solution containing the dissolved Compound 1 to a temperature of about 20-30° C. In various embodiments, the solution containing the dissolved Compound 1 is further maintained at this lower temperature for a time period, for example for about 30 min, about 1 h, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h or more.

In various embodiments, the steps of active heating followed by active cooling are repeated multiple times, for example at least 2, at least 3, at least 4, at least 5, at least 6, at least 7, at least 8, at least 9, or at least 10 times. In some embodiments, the steps of active heating followed by active cooling are repeated 2, 3, 4, 5, 6, 7, 8, 9, or 10 times.

In various embodiments, the crystallization further involves filtering the solution containing the obtained crystals of the crystalline composition comprising Compound I (e.g., comprising Formula I). In some embodiments, the crystallization optionally involves washing the obtained crystals by a solvent, for example by the recrystallization solvent one or more times. In some embodiments, the crystallization optionally involves drying the obtained crystals, for example under vacuum.

In some embodiments, the chemical purity of the crystalline composition comprising Compound I (e.g., comprising Formula I) is greater than 60%, 70%, 80%, 90%, 95%, or 99%. In some embodiments, the chemical purity of the crystalline composition comprising Compound I (e.g., comprising Formula I) is greater than about 90%. In some embodiments, the chemical purity of the crystalline composition comprising Compound I (e.g., comprising Formula I) is greater than about 95%. In some embodiments, the chemical purity of the crystalline composition comprising Compound I (e.g., comprising Formula I) is greater than about 99%. The chemical purity of the crystalline composition comprising Compound I (e.g., comprising Formula I) may be measured by any available analytical technique, for example by HPLC analysis.

In various embodiments, the crystalline composition comprising Compound I (e.g., comprising Formula I) is dry. In various embodiments, the crystalline composition comprising Compound I (e.g., comprising Formula I) is non-solvated. In various embodiments, the crystalline composition comprising Compound I (e.g., comprising Formula I) is non-hydrated. In various embodiments, the crystalline composition comprising Compound I (e.g., comprising Formula I) is anhydrous.

### Methods of Treatment

In another aspect, the present disclosure provides a method of treating or preventing testicular adrenal rest tumors (TART) or ovarian adrenal rest tumors (OART) in a subject in need thereof, comprising administering to the subject a composition comprising Compound I, or a crystalline composition comprising Compound 1 as described herein, or a pharmaceutical composition comprising the crystalline composition comprising Formula I as described herein. In some embodiments, the subject has congenital adrenal hyperplasia (CAH).

In another aspect, the present disclosure provides a method of treating congenital adrenal hyperplasia (CAH) in a subject in need thereof, comprising administering to the subject a composition comprising Compound I, or a crystalline composition comprising Compound I (e.g., comprising Formula I) as described herein, or a pharmaceutical composition comprising a crystalline composition comprising Formula I as described herein. In some embodiments, the CAH is classic CAH. In some embodiments, the CAH is non-classical CAH.

In another aspect, the present disclosure provides a method of improving hyperandrogenic symptoms in a subject in need thereof, comprising administering to the subject a composition comprising Compound I, or a crystalline composition comprising Compound I (e.g., comprising Formula I) as described herein, or a pharmaceutical composition comprising the crystalline composition comprising Formula I as described herein. In some embodiments, the hyperandrongic symptoms are selected from the group consisting of acne, hirsutism, and alopecia.

In another aspect, the present disclosure provides a method of treating menstrual irregularity, ovulatory dysfunction or infertility, in a subject in need thereof, comprising administering to the subject a composition comprising Compound I, or a crystalline composition comprising Compound I (e.g., comprising Formula I) as described herein, or a pharmaceutical composition comprising the crystalline composition comprising Formula I as described herein.

In another aspect, the present disclosure provides a method of improving metabolic symptoms in a subject in need thereof, comprising administering to the subject a a composition comprising Compound I, or crystalline composition comprising Compound I (e.g., comprising Formula I) as described herein, or the pharmaceutical composition comprising the crystalline composition comprising Formula I as described herein. In some embodiments, the metabolic symptoms are selected from the group consisting of body weight, BMI, fat mass, waist circumference, blood pressure and glycemic control.

In another aspect, the present disclosure provides a method of treating polycystic ovary syndrome with functional ovarian hyperandrogenism and functional adrenal hyperandrogenism (PCOS-FOH+FAH) in a subject in need thereof, comprising administering to the subject a composition comprising Compound I, or a crystalline composition comprising Compound I (e.g., comprising Formula I) as described herein, or the pharmaceutical composition comprising the crystalline composition comprising Formula I as described herein.

In another aspect, the present disclosure provides a method of treating polycystic ovary syndrome with functional adrenal hyperandrogenism (PCOS-FAH) in a subject in need thereof, comprising administering to the subject a composition comprising Compound I, or a crystalline composition comprising Compound I (e.g., comprising Formula I) as described herein, or the pharmaceutical composition comprising the crystalline composition comprising Compound I (e.g., comprising Formula I) as described herein.

In another aspect, the present disclosure provides a method of treating endometriosis or improving the symptoms of endometriosis in a subject in need thereof, comprising administering to the subject a composition comprising Compound I, or a crystalline composition comprising Compound I (e.g., comprising Formula I) as described herein, or the pharmaceutical composition comprising the crystalline composition comprising Formula I as described herein. In some embodiments, the administration of the crystalline composition or the pharmaceutical composition improves the symptom of pain experienced by the subject. In some embodiments, the administration of the crystalline composition or the pharmaceutical composition reduces the symptoms of pain experienced by the subject. In some embodiments, the administration of the crystalline composition or the pharmaceutical composition prevents the symptom of pain experienced by the subject. In some embodiments, the administration of the crystalline composition or the pharmaceutical composition eliminates the symptom of pain experienced by the subject.

In some embodiments, the methods described herein result in the reduction of a hormone level. Such hormones include deoxycorticosterone, 11-deoxycortisol, Cortisol, corticosterone, aldosterone, pregnenolone, hydroxy pregnenolone, progesterone, 17a-hydroxy progesterone (17-OHP), dehydroepiandrosterone, androstenediol, androstenedione, testosterone, dihydrotestosterone, estrone, estradiol, estriol, and adrenocorticotropic hormone (ACTH). In some embodiments, the methods described herein result in the reduction of 17a-hydroxy progesterone (17-OHP). In some embodiments, the methods described herein result in the reduction of adrenocorticotropic hormone (ACTH), also known as corticotropin.

In various embodiments of the methods as described herein, a composition comprising Compound I, or the crystalline composition or the pharmaceutical composition (e.g., comprising Compound 1) is administered at a dose from about 5 mg/day to about 1000 mg/day. In some embodiments, the crystalline composition or the pharmaceutical composition is administered in a fed state. In some embodiments, the crystalline composition or the pharmaceutical composition is administered in a fasted state. In some embodiments, the crystalline composition or the pharmaceutical composition is administered at least once per day.

In some embodiments of the methods described herein, the composition, or the crystalline composition or the pharmaceutical composition is administered at bedtime. In some embodiments, the methods described herein include administration of the crystalline composition or the pharmaceutical composition described herein less than about 4 hours before sleep. In some embodiments, the methods described herein include administration of the crystalline composition or the pharmaceutical composition described herein less than about 3 hours before sleep. In some embodiments, the methods described herein include administration of the crystalline composition or the pharmaceutical composition described herein less than about 2 hours before sleep. In some embodiments, the methods described herein include administration of the crystalline composition or the pharmaceutical compositions described herein less than about 1 hour before sleep. In some embodiments, the methods described herein include administration of the crystalline composition or the pharmaceutical compositions described herein less than about 30 mins before sleep.

In some embodiments, the methods described herein include administration of the crystalline composition (e.g., comprising Compound 1) or the pharmaceutical compositions (e.g., comprising Compound 1) described herein at or before the expected circadian release of adrenocorticotropic hormone (ACTH). In some embodiments, the methods described herein include administration of the crystalline composition or the pharmaceutical compositions described herein about 3-4 hours before the expected circadian release of adrenocorticotropic hormone (ACTH).

In various embodiments of the methods as described herein, the method further comprises administering to the subject an additional therapeutic agent. In some embodiments, the additional therapeutic agent is glucocorticoid, a mineralocorticoid, an ACAT1 inhibitor, or an anti-androgen agent. In some embodiments, the glucocorticoid is beclomethasone, betamethasone, budesonide, cortisone, dexamethasone, hydrocortisone, methylprednisolone, prednisolone, prednisone, or triamcinolone. In some embodiments, the mineralocorticoid is fludrocortisone. In some embodiments, the additional therapeutic agent is another CR 1antagonist.

In various embodiments of the methods as described herein, the method further comprises an additional treatment selected from surgical resection of the tumors and radiation therapy, or a combination thereof. In some embodiments, the additional therapy is surgical resection and the surgical resection is prior to, after, and/or concurrent with administration of the crystalline composition as described herein, or the pharmaceutical composition comprising the crystalline composition as described herein. In some embodiments, the additional therapy is radiation therapy and the radiation therapy is prior to, after, and/or concurrent with administration of the crystalline composition as described herein, or the pharmaceutical composition comprising the crystalline composition as described herein.

In various embodiments of the methods, the subject is from about 9 years of age to about 18 years of age. In some embodiments, the subject is from about 8 years of age to about 55 years of age.

In some aspects, described herein are crystalline compositions comprising 4-(2-chloro-4-methoxy-5-methylphenyl)-N-[(1S)-2-cyclopropyl-1-(3-fluoro-4-methylphenyl)ethyl]-5-methyl-N-(2-propynyl)-1,3-thiazol-2-amine (“crinecerfont”). In some embodiments, crinecerfont is a salt (e.g., hydrochloric acid salt, p-toluenesulfonic acid salt, etc.). Pharmaceutically acceptable acid addition salts of crinecerfont may be formed by combining a compound having a basic moiety with inorganic acids and organic acids. Inorganic acids which may be used to prepare salts of crinecerfont include, for example, hydrochloric acid, hydrobromic acid, sulfuric acid, nitric acid, phosphoric acid, and the like. Organic acids which may be used to prepare salts of crinecerfont include, for example, methanesulfonic acid, ethanesulfonic acid, p-toluenesulfonic acid, and the like. Pharmaceutically acceptable base addition salts of crinecerfont may be formed by combining a compound having an acidic moiety with inorganic and organic bases. Inorganic bases which may be used to prepare salts of crinecerfont include, for example, sodium, potassium, lithium, ammonium, calcium, magnesium, iron, zinc, manganese, aluminum hydroxides, carbonates, bicarbonates, phosphates, and the like. In some embodiments, the inorganic base salt is ammonium, potassium, sodium, calcium, and magnesium hydroxides, carbonates, bicarbonates, or phosphates. Organic bases from which may be used to prepare salts of crinecerfont include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Generally, such salts of crinecerfont may be prepared by reacting the free acid or base forms of these compounds with at least a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN). In some embodiments, they crystalline composition comprises crinecerfont and oxalic acid.

In some embodiments, the crystalline composition comprising crinecerfont comprises crinecerfont and an acid in a ratio of crinecerfont to acid of about 2:1, about 1:1, or about 1:2.

In some embodiments, the composition comprising crinecerfont is in a capsule (e.g., an immediate-release capsule or a capsule for opening (e.g., comprising crinecerfont granules).

In some aspects, disclosed herein are methods of administering a composition (e.g., crystalline composition) comprising crinecerfont. In some embodiments, the method further comprises administering an additional therapeutic. In some embodiments, the additional therapeutic. In some embodiments, the additional therapeutic comprises hydrocortisone. In some embodiments, the hydrocortisone is an immediate-release hydrocortisone. In some embodiments, the hydrocortisone is in a capsule (e.g., a capsule for opening (e.g., hydrocortisone granules in a capsule)). In some embodiments, the hydrocortisone is an modified-release hydrocortisone. In some embodiments, the hydrocortisone is in a hard-capsule (e.g., a modified-release hard capsule).

### Additional Embodiments

The following embodiments are given for the purpose of illustrating various embodiments of the disclosure and are not meant to limit the present disclosure or any aspect thereof (e.g., element) in any fashion. The following embodiments may include additional elements disclosed herein. Changes therein and other uses which are encompassed within the spirit of the disclosure as defined by the scope of the claims will occur to those skilled in the art.

1. A pharmaceutical formulation, comprising:
(a) about 10 mg to about 300 mg of 3-(4-Chloro-2-(morpholin-4-yl)thiazol-5-yl)-7-(1-ethylpropyl)-2,5-dimethylpyrazolo(1,5-α) pyrimidine (Compound 1), or a pharmaceutically acceptable salt or solvate thereof;
(b) a first diluent in about 40% to about 45% by weight, wherein the first diluent comprises lactose monohydrate;
(c) a second diluent in about 25% by weight, wherein the second diluent comprises microcrystalline cellulose;
(d) about 1% to about 6% by weight of hydroxypropyl cellulose;
(e) about 0.5% to about 5% by weight of sodium lauryl sulfate; and
(f) about 4% by weight of croscarmellose sodium.

2. The pharmaceutical formulation of embodiment 1, further comprising a glidant, wherein the glidant comprises colloidal silicon dioxide.

3. The pharmaceutical formulation of embodiment 1, further comprising a lubricant, wherein the lubricant comprises magnesium stearate.

4. The pharmaceutical formulation of embodiment 3, wherein the magnesium stearate is about 1% by weight of the pharmaceutical formulation.

5. The pharmaceutical formulation of embodiment 3, wherein the magnesium stearate is in an amount that is about 2 mg.

6. The pharmaceutical formulation of embodiment 1, wherein Compound 1 or a pharmaceutically acceptable salt thereof is in an amount that is about 50 mg.

7. The pharmaceutical formulation of embodiment 1, wherein the lactose monohydrate is about 40.25% by weight of the pharmaceutical formulation.

8. The pharmaceutical formulation of embodiment 1, wherein the lactose monohydrate is in an amount that is about 80.50 mg.

9. The pharmaceutical formulation of embodiment 1, wherein the microcrystalline cellulose is in an amount that is about 50 mg.

10. The pharmaceutical formulation of embodiment 1, wherein the hydroxypropyl cellulose is about 3% by weight of the pharmaceutical formulation.

11. The pharmaceutical formulation of embodiment 1, wherein the hydroxypropyl cellulose is in an amount that is about 6 mg.

12. The pharmaceutical formulation of embodiment 1, wherein the sodium lauryl sulfate is about 0.75% by weight of the pharmaceutical formulation.

13. The pharmaceutical formulation of embodiment 1, wherein the sodium lauryl sulfate is in an amount that is about 1.50 mg.

14. The pharmaceutical formulation of embodiment 1, wherein the croscarmellose sodium is in an amount that is about 8 mg.

15. The pharmaceutical formulation of embodiment 2, wherein the colloidal silicon dioxide is about 1% by weight of the pharmaceutical formulation.

16. The pharmaceutical formulation of embodiment 2, wherein the colloidal silicon dioxide is in an amount that is about 2 mg.

17. The pharmaceutical formulation of embodiment 3, wherein the magnesium stearate is about 1% by weight of the pharmaceutical formulation.

18. The pharmaceutical formulation of embodiment 3, wherein the magnesium stearate is in an amount that is about 2 mg.

19. The pharmaceutical formulation of embodiment 1, wherein the pharmaceutical formulation is in a tablet form.

20. The pharmaceutical formulation of embodiment 1, wherein the pharmaceutical formulation is in a granule form.

21. The pharmaceutical formulation of embodiment 1, wherein the pharmaceutical formulation is in a capsule.

22. The pharmaceutical formulation of embodiment 1, wherein the pharmaceutical formulation is packaged in a sachet.

### Examples

The following examples serve to further describe the manner of using the present disclosure. These examples are presented for illustrative purpose and should not serve to limit the true scope of the present disclosure.

In carrying out the procedures of the methods described herein, it is of course to be understood that references to particular buffers, media, reagents, cells, culture conditions, and the like are not intended to be limiting, but are to be read so as to include all related materials that one of ordinary skill in the art would recognize as being of interest or value in the particular context in which that discussion is presented. For example, it is often possible to substitute on buffer system or culture medium for another and still achieve similar, if not identical, results.

Those of skill in the art will have sufficient knowledge of such systems and methodologies so as to able, without undue experimentation, to make such substitutions as will optimally serve their purposes in using the methods and procedures disclosed herein.

### Example 1—Preparation and Characterization of the Crystalline Composition Comprising Compound I (e.g., comprising Formula I)

Preparation of the Crystalline Composition Comprising Compound I (e.g., comprising Formula I)

A mixture of 185.0 mg (0.441 mmol) of 4-(4-chloro-5-(2,5-dimethyl-7-(pentan-3-yl)pyrazolo[1,5-a]pyrimidin-3-yl)thiazol-2-yl)morpholine (Compound 1), 45.0 mg (0.500 mmol) of oxalic acid, and 2.5 mL of diisopropyl ether was agitated. After one hour, an additional 1 mL of diisopropyl ether was added. After stirring overnight, the mixture was vacuum filtered and the resulting solid was dried to afford 170 mg (76%) of the crystalline composition comprising Compound I (e.g., comprising Formula I) as solid.

### X-Ray Powder Diffraction

X-ray powder diffraction (XPRD) patterns were obtained on a Rigaku Smart-Lab X-ray diffraction system. A CuK source (=0.71073 Å) operating minimally at 40 kV and 44 mA scans each sample between 2 and 40 degrees 2θ. The step size is 0.02° 2θ.

The XRPD pattern obtained for the crystalline composition comprising Compound I (e.g., comprising Formula I) is summarized in Table 1 below and shown in FIG. 1.

TABLE 1
XRPD Data of the Crystalline Composition Comprising
Compound I (e.g., comprising Formula I)

| Peak # | Angle (° 2-θ) | Intensity (%) |
|---|---|---|
| 1 | 5.44 | 31 |
| 2 | 8.82 | 12 |
| 3 | 10.84 | 100 |
| 4 | 11.70 | 12 |
| 5 | 14.60 | 8 |
| 6 | 15.56 | 6 |
| 7 | 15.96 | 52 |
| 8 | 16.28 | 5 |
| 9 | 16.70 | 10 |
| 10 | 17.66 | 4 |
| 11 | 18.44 | 5 |
| 12 | 18.82 | 6 |
| 13 | 19.18 | 8 |
| 14 | 19.60 | 2 |
| 15 | 20.02 | 11 |
| 16 | 20.50 | 11 |
| 17 | 20.78 | 20 |
| 18 | 21.72 | 7 |
| 19 | 22.12 | 2 |
| 20 | 22.74 | 18 |
| 21 | 23.04 | 16 |
| 22 | 23.44 | 31 |
| 23 | 24.80 | 48 |
| 24 | 25.52 | 6 |
| 25 | 25.92 | 9 |
| 26 | 26.58 | 4 |
| 27 | 26.80 | 14 |
| 28 | 26.94 | 8 |
| 29 | 27.62 | 3 |
| 30 | 28.00 | 13 |
| 31 | 28.86 | 14 |
| 32 | 29.54 | 3 |
| 33 | 31.26 | 3 |
| 34 | 31.66 | 5 |
| 35 | 31.88 | 4 |
| 36 | 32.16 | 3 |
| 37 | 32.48 | 2 |
| 38 | 32.86 | 5 |
| 39 | 33.46 | 2 |
| 40 | 34.36 | 2 |
| 41 | 35.02 | 3 |
| 42 | 35.44 | 1 |
| 43 | 35.98 | 2 |
| 44 | 36.72 | 4 |
| 45 | 37.04 | 3 |
| 46 | 37.80 | 5 |
| 47 | 38.28 | 2 |
| 48 | 38.90 | 2 |
| 49 | 39.20 | 2 |

### Differential Scanning Calorimetry (DSC)

Differential scanning calorimetry analysis was carried out on a TA Instruments Q2500 Discovery Series instrument. The instrument temperature calibration was performed using indium. The DSC cell was kept under a nitrogen purge of about 50 mL per minute during each analysis. The sample was placed in a standard, crimpled, aluminum pan and was heated from approximately 25° C. to 350° C. at a rate of 10° C. per minute. The DSC trace of the crystalline composition comprising Compound I (e.g., comprising Formula I) is shown in FIG. 2.

### Thermogravimetric Analysis (TGA)

Thermogravimetry analyses were carried out using a TA Instruments Discovery TGA 5500 instrument that was cooled using a TA Instruments Refrigerated Cooling System (RCS) 90 chiller. The instrument balance was calibrated using class M weights and temperature calibration was performed by measurement of the Curie point of Alumel®. Sample was loaded onto a platinum sample pan and the pan was loaded into the TG instrument. The pan was heated from ambient temperature to 350° C. at a rate of 10° C. per minute. The instrument was controlled using TA Trios software. The TGA trace of the crystalline composition comprising Compound I (e.g., comprising Formula I) is shown in FIG. 3.

### Dynamic Vapor Sorption (DVS)

The DVS analysis was carried out on a TA Instruments Q5000 sorption analyzer. The instrument was calibrated with standard weights and a sodium bromide standard for humidity. For each analysis, a portion of the sample was weighed into a metal-coated, quartz pan. Each sample was analyzed at 25° C. with a maximum equilibration time of 60 minutes in 10% relative humidity (RH) steps from 5 to 95% RH (adsorption cycle) and from 95 to 5% RH (desorption cycle). Data collection was performed using Advantage for Q Series version 5.5.23. The percent weight change values were calculated using Microsoft Excel®. The DVS trace of the crystalline composition comprising Compound I (e.g., comprising Formula I) is shown in FIG. 4.

### Infrared (IR)

The IR spectrum was obtained using a Nicolet iS50 Model 60825 Fourier-transform (FT) IR spectrophotometer equipped with a deuterated triglycine sulfate (DTGS) detector, a potassium bromide (KBr) beamsplitter, and an electronically temperature controlled (ETC) Ever-Glo® IR source. The instrument was configured with a SMART iTR diamond attenuated total reflectance (ATR) sampling accessory. The single beam scan of the background (air) and sample were collected with 128 signal-averaged scans at a resolution of 2 cm-1 over the spectral range 400-4000 cm-1. The wavelength calibration was verified using a certified polystyrene standard. Data collection and processing was performed using Omnic 9.2 software. The IR spectrum of the crystalline composition comprising Compound I (e.g., comprising Formula I) is shown in FIG. 5.

### Proton Nuclear Magnetic Resonance (¹H NMR)

The 1H NMR spectrum was acquired on a Bruker Avance 400 MHz spectrometer using TopSpin v3.2 software. The sample was dissolved in CD₃OD and the resulting solution was transferred into a 5-mm NMR tube for subsequent data acquisition. Data collection parameters are shown in Table 14. The spectrum was processed using the program MNova and referenced to the chemical shift of the residual protons in CD₃OD (3.31 ppm). The ¹H NMR spectrum of the crystalline composition comprising Compound I (e.g., comprising Formula I) is shown in FIG. 6.

### Raman

The Raman spectrum was acquired utilizing a Thermo Scientific model iS50 Fourier-transform (FT) IR spectrophotometer equipped with a Raman accessory. The system is equipped with an indium gallium arsenide (InGaAs) detector, a calcium fluoride (CaF2) beamsplitter, and a 2.5 W laser operating at 1064 nm. The module was configured with a 180° reflective sampling accessory. A sample was prepared by loading particles onto a glass slide. Each FT-Raman spectrum was collected using 0.50 W laser power with 256 signal-averaged scans at a resolution of 4 cm-1 over the spectral range 100-3700 cm-1. The wavelength calibration was verified using a polystyrene. Data acquisition and processing were performed using Omnic 9.7 software. The Raman spectrum of the crystalline composition comprising Compound I (e.g., comprising Formula I) is shown in FIG. 7.

### Example 2—Formulation Comprising Compound 1

### General Manufacturing Procedure

The relevant excipients were manually sieved and then dry mixed. A binder solution was added and the mixture subjected to wet granulation. The granules were then wet milled and dried. The resulting granules were dry milled and added to other relevant common granules and excipients. The mixture was then compressed into tablets to a target tablet weight.

A granulation was developed with a minimum amount and number of excipients. The granulation comprised of Compound 1 (“Tildacerfont”) with a surfactant and a binder and, in some cases, a super-disintegrant. After granulation was successful, other excipients were added to enable compression. These excipients included a compression aid, a lubricant, an anti-tacking agent, and a super-disintegrant.

Table 1 below summarizes formulations comprising Tildacerfont in both tablet and granules form.

TABLE 2
Formulation Comprising Tildacerfont

| | | | | |
|---|---|---|---|---|
| | | | mg/Tablet | Granules |
| | | | 50 mg | 50 mg |
| Material Description | Function | % w/w | strength | strength |

Intragranular

| | | | | |
|---|---|---|---|---|
| Tildacerfont (SPN001) | Active | 25.00 | 50.00 | 50.00 |
| | ingredient | | | |
| Lactose Monohydrate, | Diluent | 40.25 | 80.50 | 80.50 |
| USP/NF, Ph. Eur., JP. | | | | |
| (Pharmatose 200M EU) | | | | |
| Hydroxypropylcellulose | Binder | 3.00 | 6.00 | 6.00 |
| USP/NF, Ph. Eur., JP | | | | |
| (Klucel EXF) | | | | |
| Sodium Lauryl Sulfate, | Surfactant | 0.75 | 1.50 | 1.50 |
| NF/ Ph. Eur./JP | | | | |
| (Kolliphor SLS fine) | | | | |
| Microcrystalline | Diluent | 20.00 | 40.00 | 40.00 |
| Cellulose, NF, Ph. Eur., | | | | |
| JP (Avicel PH101) | | | | |
| Purified Water, USP, | Granulating | QS* | — | — |
| Ph. Eur | agent | | | |

Extragranular

| | | | | |
|---|---|---|---|---|
| Microcrystalline | Diluent | 5.00 | 10.00 | 10.00 |
| Cellulose, NF, Ph. Eur., | | | | |
| JP (Avicel PH101) | | | | |
| Croscarmellose Sodium | Disintegrant | 4.00 | 8.00 | 8.00 |
| NF, Ph. Eur., JP | | | | |
| (Ac-Di-Sol) SD-711 | | | | |
| Colloidal Silicon dioxide | Glidant | 1.00 | 2.00 | 2.00 |
| USP/NF, Ph. Eur., JP | | | | |
| (Aerosil 200) | | | | |
| Magnesium stearate, | Lubricant | 1.00 | 2.00 | 2.00 |
| NF, Ph. Eur., JP | | | | |
| (Hyqual 2257) | | | | |

| | | | |
|---|---|---|---|
| Total | 100.00 | 200.00 | 200.00 |

Film Coating

| | | | | |
|---|---|---|---|---|
| Opadry II Yellow | Film former | 3.00 | 6.00 | — |
| 85F42129 | | | | |
| Purified Water, USP, | Coating | QS* | — | — |
| Ph. Eur | dispersion | | | |
| | medium | | | |

| | | | |
|---|---|---|---|
| Total | 100.00 | 206.00 | 200.00 |

### Example 3—Stability of Crystalline Compositions Comprising Compound 1

To determine the stability of the crystalline compositions prepared comprising the methods described herein, crystalline compositions comprising Compound 1 and oxalic acid were formulated. Samples of the crystalline composition comprising Compound 1-oxalic acid co-crystal were packed in a transparent low-density polyethylene (LDPE) bag, and closed by twisting the bag and tying it with a strip seal. The transparent LDPE bag was then placed in a black LDPE bag that was twisted and tied with a strip seal. The bags were then placed in a high-density polyethylene container.

The samples were then subjected to accelerated aging conditions (40±2° C./75±5% relative humidity (RH)) or long-term storage conditions (25±2° C./60±5% RH). Sample stability measurements in each condition were measured at baseline (before exposure to accelerated aging conditions or long-term storage conditions), and one month, two months, and three months after exposure to said conditions. Sample stability measurements included a description of the sample's physical appearance (e.g., color and form), identification by Fourier-transform infrared spectroscopy (FT-IR), identification by X-ray powder diffraction (XRPD), water content (measured by Karl Fischer (KF) on a % weight/weight basis), identification of related substances (e.g., impurities), and high-performance liquid chromatography ((HPLC) measured on a % weight/weight on an anhydrous basis and solvent free basis).

Table 3 summarizes the results of the measurements obtained from samples exposed to the accelerated aging conditions over the course of one to three months. Table 4 summarizes the results of the study obtained from the samples exposed to the long-term storage conditions over the course of one to three months.

TABLE 3
Stability data under accelerated aging conditions.

| Parameter | Specification | Baseline | 1 Month | 2 Months | 3 Months |
|---|---|---|---|---|---|
| Description | Off-white to | Light yellow | Light yellow | Light yellow | Light yellow |
| | yellow color | color solid | color solid | color solid | color solid |
| | solid | | | | |
| Identification | IR spectrum | IR spectrum | IR spectrum | IR spectrum | IR spectrum |
| by FT-IR | of sample | of sample is | of sample is | of sample is | of sample is |
| | should be | concordant | concordant | concordant | concordant |
| | concordant | with that of | with that of | with that of | with that of |
| | with that of | reference | reference | reference | reference |
| | reference | standard | standard | standard | standard |
| | standard | | | | |
| Identification | Diffractogram | Diffractogram | Diffractogram | Diffractogram | Diffractogram |
| by XRPD | of the sample | of the sample | of the sample | of the sample | of the sample |
| | should be | is consistent | is consistent | is consistent | is consistent |
| | consistent with | with | with | with | with |
| | characteristic | characteristic | characteristic | characteristic | characteristic |
| | 2-θ value of | 2-θ value of | 2-θ value of | 2-θ value of | 2-θ value of |
| | Form-I at 5.4, | Form-I at 5.4, | Form-I at 5.4, | Form-I at 5.4, | Form-I at 5.4, |
| | 8.8, 10.8, | 8.8, 10.8, | 8.8, 10.8, | 8.8, 10.8, | 8.8, 10.8, |
| | 11.6, 15.9, | 11.6, 15.9, | 11.7, 15.9, | 11.6, 15.9, | 11.6, 15.9, |
| | 23.4 ± 0.2° | 23.4 | 23.4 | 23.4 | 23.4 |
| Water Content | NMT 0.5 | 0.02 | 0.09 | 0.02 | 0.08 |
| by KF (% w/w) | | | | | |
| Related Substances | | | | | |
| by HPLC (% area) | | | | | |
| (see a.-d.) | | | | | |
| a. | NMT 0.15 | BDRL | BDRL | BDRL | BDRL |
| C07102401-F-Imp1 | | | | | |
| b. | NMT 0.15 | BDRL | BDRL | BDRL | BDRL |
| C07102401-F-Imp2 | | | | | |
| c. Any unspecified | NMT 0.10 | BDRL | BDRL | BDRL | BDRL |
| impurity | | | | | |
| d. Total impurities | NMT 1.0 | BDRL | BDRL | BDRL | BDRL |
| Assay of Compound | NLT 98.0 and | 99.9 | 99.2 | 100.1 | 99.2 |
| 1 by HPLC | NMT 102.0 | | | | |
| (% w/w on anhydrous | | | | | |
| basis and solvent | | | | | |
| free basis) | | | | | |

n/a: not applicable;
NLT: not less than;
NMT; not more than;
BDRL: below disregard limit

The above data from Table 3 indicates that the Compound 1-oxalic acid co-crystal is stable up to 3 months at accelerated aging conditions (40±2° C./75±5% RH).

TABLE 4
Stability data under long-term storage conditions.

| Parameter | Specification | Baseline | 1 Month | 2 Months | 3 Months |
|---|---|---|---|---|---|
| Description | Off-white to | Light yellow | Light yellow | Light yellow | Light yellow |
| | yellow color | color solid | color solid | color solid | color solid |
| | solid | | | | |
| Identification | IR spectrum | IR spectrum | IR spectrum | IR spectrum | IR spectrum |
| by FT-IR | of sample | of sample is | of sample is | of sample is | of sample is |
| | should be | concordant | concordant | concordant | concordant |
| | concordant | with that of | with that of | with that of | with that of |
| | with that of | reference | reference | reference | reference |
| | reference | standard | standard | standard | standard |
| | standard | | | | |
| Identification | Diffractogram | Diffractogram | Diffractogram | Diffractogram | Diffractogram |
| by XRPD | of the sample | of the sample | of the sample | of the sample | of the sample |
| | should be | is consistent | is consistent | is consistent | is consistent |
| | consistent with | with | with | with | with |
| | characteristic | characteristic | characteristic | characteristic | characteristic |
| | 2-θ value of | 2-θ value of | 2-θ value of | 2-θ value of | 2-θ value of |
| | Form-I at 5.4, | Form-I at 5.4, | Form-I at 5.4, | Form-I at 5.4, | Form-I at 5.4, |
| | 8.8, 10.8, | 8.8, 10.8, | 838, 10.8, | 8.7, 10.8, | 837, 10.8, |
| | 11.6, 15.9, | 11.6, 15.9, | 11.7, 15.9, | 11.6, 15.9, | 11.6, 15.9, |
| | 23.4 ± 0.2° | 23.4 | 23.4 | 23.3 | 23.4 |
| Water Content | NMT 0.5 | 0.02 | 0.10 | 0.03 | 0.05 |
| by KF (% w/w) | | | | | |
| Related Substances | | | | | |
| by HPLC (% area) | | | | | |
| (see a.-d.) | | | | | |
| a. | NMT 0.15 | BDRL | BDRL | BDRL | BDRL |
| C07102401-F-Imp1 | | | | | |
| b. | NMT 0.15 | BDRL | BDRL | BDRL | BDRL |
| C07102401-F-Imp2 | | | | | |
| c. Any unspecified | NMT 0.10 | BDRL | BDRL | BDRL | BDRL |
| impurity | | | | | |
| d. Total impurities | NMT 1.0 | BDRL | BDRL | BDRL | BDRL |
| Assay of Compound | NLT 98.0 and | 99.9 | 99.2 | 100.1 | 99.2 |
| 1 by HPLC | NMT 102.0 | | | | |
| (% w/w on anhydrous | | | | | |
| basis and solvent | | | | | |
| free basis) | | | | | |

n/a: not applicable;
NLT: not less than;
NMT; not more than;
BDRL: below disregard limit

The above data from Table 4 indicates that the Compound 1-oxalic acid co-crystal is stable up to 3 months at long-term storage conditions (25±2° C./60±5% RH).

### Example 4—Stability of Crystalline Compositions Comprising Compound 1 for 6 Months

Samples of the crystalline composition comprising Compound 1-oxalic acid co-crystal prepared in Example 3 are subjected to accelerated aging conditions (40±2° C./75±5% relative humidity (RH)) or long-term storage conditions (25±2° C./60±5% RH). Sample stability measurements in each condition are measured at baseline (before exposure to accelerated aging conditions or long-term storage conditions), and four months, five months, six months, seven months, eight months, nine months, ten months, eleven months, twelve months, fourteen months, sixteen months, and eighteen months after exposure to said conditions. Sample stability measurements include a description of the sample's physical appearance (e.g., color and form), identification by Fourier-transform infrared spectroscopy (FT-IR), identification by X-ray powder diffraction (XRPD), water content (measured by Karl Fischer (KF) on a % weight/weight basis), identification of related substances (e.g., impurities), and high-performance liquid chromatography ((HPLC) measured on a % weight/weight on an anhydrous basis and solvent free basis).

The samples of the crystalline composition comprising Compound 1-oxalic acid co-crystal will be stable at four months, five months, six months, seven months, eight months, nine months, ten months, eleven months, twelve months, fourteen months, sixteen months, and eighteen months after exposure to said conditions, as demonstrated by sample stability measurements. Said sample stability measurements at four months, five months, six months, seven months, eight months, nine months, ten months, eleven months, twelve months, fourteen months, sixteen months, and eighteen months after exposure to said conditions will be in accordance with the results listed in Table 3 and Table 4.

1. A pharmaceutical composition comprising a CRF1 antagonist, wherein the composition is characterized by an X-ray powder diffraction pattern comprising a peak selected from any one or more of 10.84±0.2° 2-θ, 15.96±0.2° 2-θ, 23.44±0.2° 2-θ, and 24.80±0.2° 2-θ, wherein the CRF1 antagonist comprises Compound 1: 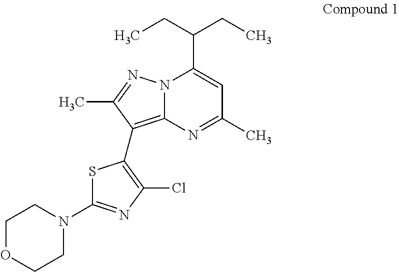 [SMILES: CCC(CC)c1cc(C)nc2c(-c3sc(N4CCOCC4)nc3Cl)c(C)nn12]
or a pharmaceutically acceptable salt thereof, and at least one pharmaceutically acceptable excipient.

2. The pharmaceutical composition of claim 1, wherein the composition is measured by X-ray powder diffraction using an X-ray wavelength of 0.71073 Å.

3. The pharmaceutical composition of claim 1, wherein the X-ray powder diffraction pattern further comprising at least one peak selected from 5.44±0.2° 2-θ, 20.78±0.2° 2-θ, 22.74±0.2° 2-θ, 23.04±0.2° 2-θ, 26.80±0.2° 2-θ, and 28.86 ±0.2° 2-θ, and as measured by X-ray powder diffraction using an X-ray wavelength of 0.71073 Å.

4. The pharmaceutical composition of claim 1, wherein the X-ray powder diffraction pattern further comprising at least five peaks selected from 8.82±0.2° 2-θ, 11.70±0.2° 2-θ, 14.60±0.2° 2-θ, 15.56±0.2° 2-θ, 16.70±0.2° 2-θ, 18.82±0.2° 2-θ, 19.18±0.2° 2-θ, 20.02±0.2° 2-θ, 20.50±0.2° 2-θ, 21.72±0.2° 2-θ, 25.52±0.2° 2-θ, 25.92±0.2° 2-θ, 26.94±0.2° 2-θ, and 28.00±0.2° 2-θ, and as measured by X-ray powder diffraction using an X-ray wavelength of 0.71073 Å.

5. The pharmaceutical composition of claim 1, wherein the pharmaceutical composition is further characterized by a thermogravimetric analysis (TGA) thermogram comprising a loss in mass of about 11% over a temperature range of about 25° C. to about 200° C.

6. The pharmaceutical composition of claim 1, wherein the pharmaceutical composition is further characterized by a thermogravimetric analysis (TGA) thermogram substantially similar to the one set forth in FIG. 2.

7. (canceled)

8. The pharmaceutical composition of claim 1, wherein the pharmaceutical composition is a crystalline pharmaceutical composition.

9. The pharmaceutical composition of claim 8, wherein the crystalline pharmaceutical composition further comprises oxalic acid.

10. The pharmaceutical composition of claim 9, wherein the crystalline pharmaceutical composition comprises Compound 1 and oxalic acid in a molar ratio of about 2:1.

11.-18. (canceled)

19. A method of preparing a crystalline composition comprising Compound 1: 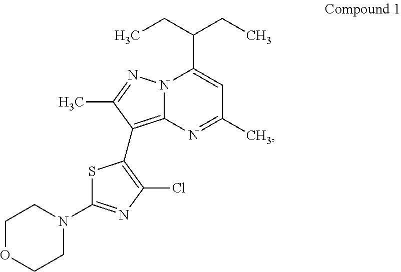 [SMILES: CCC(CC)c1cc(C)nc2c(-c3sc(N4CCOCC4)nc3Cl)c(C)nn12]
wherein the method comprises:
(a) dissolving Compound 1 and an acid in a solvent;
(b) heating the solution or slurry from step (a); and
(c) crystallizing the solution or slurry obtained in step (b) to obtain the crystalline composition comprising Compound 1.

20. The method of claim 19, wherein the acid comprises oxalic acid.

21. The method of claim 19, wherein the solvent in step (a) is heptane.

22. The method of claim 19, wherein the solvent in step (a) is methyl tert-butyl ether or methyl ethyl ketone.

23. The method of claim 19, wherein the solvent in step (a) is a mixture of heptane and methyl ethyl ketone.

24. The method of claim 19, wherein step (b) is heated at about 50° C.

25.-55. (canceled)

56. A crystalline composition comprising Formula I:

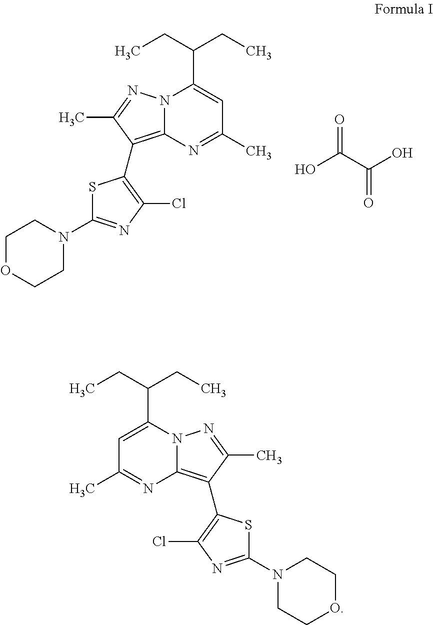

57. The crystalline composition of claim 56, wherein the crystals have unit cell parameters at T=150° K of: a=34.003(4) Å, b=6.5843(13) Å, c=21.062(5) Å; R=108.703(12°), V=4466.5(15) Å³ and a monoclinic C2/c space group.

58. The crystalline composition of claim 56, wherein the crystalline composition is characterized by:
(a) an X-ray powder diffraction pattern comprising peaks at 10.84±0.2° 2-θ, 15.96±0.2° 2-θ, 23.44±0.2° 2-θ, and 24.80±0.2° 2-θ, and as measured by X-ray powder diffraction using an X-ray wavelength of 0.71073 Å;
(b) an X-ray powder diffraction pattern substantially the same as shown in FIG. 1;
(c) a differential scanning calorimetry (DSC) thermogram comprising an endotherm in the range of about 160° C. to 170° C.;
(d) a differential scanning calorimetry (DSC) thermogram comprising an endotherm with an onset of about 164° C. and a peak of about 166° C.;
(e) a differential scanning calorimetry (DSC) thermogram substantially the same as shown in FIG. 2;
(f) a thermogravimetric analysis (TGA) thermogram comprising a loss in mass of about 11% over a temperature range of about 25° C. to about 200° C.;
(g) a thermogravimetric analysis (TGA) thermogram substantially the same as shown in FIG. 3; or
(h) combinations thereof.

59.-68. (canceled)

69. A method of preparing a crystalline composition comprising Formula I: 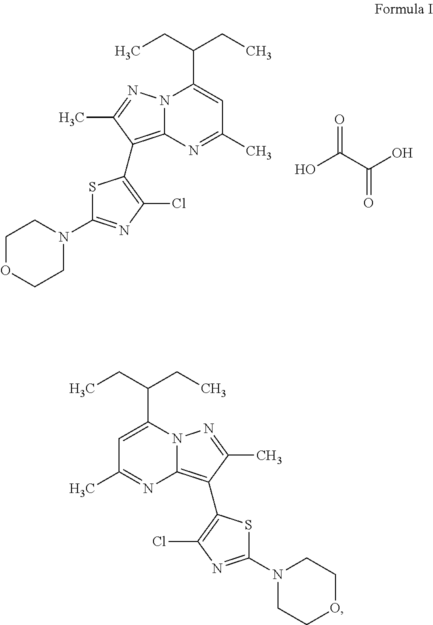
wherein the method comprises:
(a) dissolving 4-(4-chloro-5-(2,5-dimethyl-7-(pentan-3-yl)pyrazolo[1,5-a]pyrimidin-3-yl)thiazol-2-yl)morpholine (Compound 1) and oxalic acid in a solvent;
(b) heating the solution or slurry from step (a); and
(c) crystallizing the solution or slurry obtained in step (b) to obtain the crystalline composition comprising Formula I.

70. The method of claim 69, wherein the solvent in step (a) comprises acetone, heptane, water, 2-butanol, ethyl acetate, 2-propanol, methyl tert-butyl ether, ethanol, methyl ethyl ketone, 1-pentanol, or a combination thereof.

71.-105. (canceled)
